(19) United States
(12) Patent Application Publication
(10) Pub. No.: US 2006/0153839 A1
(43) Pub. Date: Jul. 13, 2006

(54) Production of bispecific molecules using polyethylene glycol linkers

(75) Inventors: Nehal Mohamed, Bud Lake, NJ (US); Leslie Casey, Newyork, NY (US); James P Porter, Sparta, NJ (US); Xiaoliang Wang, Pine Brook, NJ (US); Muctarr Sesay, Margate, FL (US); Lihsyng Stanford Lee, Bridgewater, NJ (US)

Correspondence Address: JONES DAY, 222 EAST 41ST ST, NEW YORK, NY, 10017, US

(73) Assignee: EluSys Therapeutics, Inc., Pine Brook, NJ (US)

(21) Appl. No.: 10/527,936

(22) PCT Filed: Sep. 16, 2003
(86) PCT No.: PCT/US03/29059

§ 371 Date: Feb. 28, 2006

Related U.S. Application Data
(60) Provisional application No. 60/411,731, filed on Sep. 16, 2002.

(51) Int. Cl. A61K 39/40; C07K 16/12

(52) U.S. Cl. 424/143.1; 424/164.1; 530/388.4

(57) ABSTRACT

The invention relates to a bispecific molecule comprising a first recognition binding moeity that binds a Cab-like receptor cross-linked using a poly-(ethylene glycol) (“PEG”) linker with one or more second recognition binding moieties that bind a molecule. The invention also relates to methods of producing such bispecific molecules and to therapeutic uses of such bispecific molecules.

This application claims the benefit of U.S. Application Ser. No. 60/411,731 filed on Sep. 16, 2002 which is incorporated herein by reference in its entirety.

### 1. FIELD OF THE INVENTION

The invention relates to a bispecific molecule comprising a first recognition binding moeity that binds a C3b-like receptor cross-linked using a poly-(ethylene glycol) (“PEG”) linker with one or more second recognition binding moieties that bind a molecule. The invention also relates to methods of producing such bispecific molecules and to therapeutic uses of such bispecific molecules.

### 2. BACKGROUND OF THE INVENTION

Primate erythrocytes, or red blood cells (RBC's), play an essential role in the clearance of antigens from the circulatory system. The formation of an immune complex in the circulatory system activates the complement factor C3b in primates and leads to the binding of C3b to the immune complex. The C3b/immune complex then binds to the type 1 complement receptor (CR1), a C3b receptor, expressed on the surface of erythrocytes via the C3b molecule attached to the immune complex. The immune complex is then chaperoned by the erythrocyte to the reticuloendothelial system (RES) in the liver and spleen for neutralization. The RES cells, most notably the fixed-tissue macrophages in the liver called Kupffer cells, recognize the C3b/immune complex and break this complex from the RBC by severing the C3b receptor-RBC junction, producing a liberated erythrocyte and a C3b/immune complex which is then engulfed by the Kupffer cells and is completely destroyed within subcellular organelles of the Kupffer cells. This pathogen clearance process, however, is complement-dependent, Le., confined to immune complexes recognized by the C3b receptor, and is ineffective in removing immune complexes which are not recognized by the C3b receptor.

Taylor et al. have discovered a complement independent method of removing pathogens from the circulatory system. Taylor et al. have shown that chemical crosslinking of a first monoclonal antibody (mAb) specific to a primate C3b receptor to a second monoclonal antibody specific to a pathogenic antigenic molecule creates a bispecific heteropolymeric antibody which offers a mechanism for binding a pathogenic antigenic molecule to a primate's C3b receptor without complement activation. (U.S. Pat. Nos. 5,487,890; 5,470,570; and 5,879,679). It is found that the Fc portion of the mAb specific to C3b receptor plays an important role in the transfer of the erythrocyte-immune complex to an acceptor cell and the subsequent proteolysis of the erythrocyte-immune complex (Nardin et al., 1999, Molecular Immunology 36:827-835). Taylor et al. have shown that this complement-independent process can remove over 99% of pathogens from the circulation as compared to about 10-15% by the normal, complement-dependent, process. Taylor also reported a HP which can be used to remove a pathogenic antigen specific autoantibody from the circulation. Such a HP, also referred to as an “Antigen-based Heteropolymer” (AHP), contains a CR1 specific monoclonal antibody cross-linked to an antigen (see, e.g., U.S. Pat. No. 5,879,679; Lindorfer, et al., 2001, Immunol Rev.183: 10-24; Lindorfer, et al., 2001, J Immunol Methods 248: 125-138; Ferguson, et. al., 1995, Arthritis Rheum 38: 190-200).

The Taylor method, however, has certain shortcomings. Firstly, the chemistry of the cross-linking reaction is not very efficient. Typically, the yields of such chemical cross-linking reactions are only about 10% to 20%. As a result, a significant amount of purified mAbs or pathogen-binding moieties is lost during the chemical cross-linking step of the manufacturing process. For example, using standard chemical cross-linking agents (such as Pierce's SATA and sulfo-SMCC), using 1 mg of pure mAb1 cross-linked to 1 mg of pure mAb2, we have generated only between 0.2 to 0.4 mg of pure product mAb1×mAb2. Secondly, the bispecific molecule produced by chemical cross-linking contains a chemical cross-linker fragment which can be immunogenic. The immunogenicity of the cross-linker can be disadvantageous when re-administering Taylor's bispecific molecule to the same individual because the individual may generate an immune response against the cross-linker moiety and, upon re-exposure of the same individual to another dose of the bispecific molecule, the individual might mount a vigorous immune response against it, reducing therapeutic benefits that the bispecific molecule would otherwise provide. Thirdly, the cross-linking process described in the Taylor patents is not site-specific, and consequently, may decrease somewhat the functionality of the mAbs or pathogen recognition domains. Therefore, there is a need for a more efficient method for the production of bispecific molecules.

Discussion or citation of a reference herein shall not be construed as an admission that such a reference is a prior art to the present invention.

### 3. SUMMARY OF THE INVENTION

The present invention relates to bispecific molecules comprising a first recognition binding moiety which binds a C3b-like receptor or a functional equivalent thereof (known as complement receptor 1 (CR1) or CD35 in primates) cross-linked using a poylethylene glycol linker to one or more second recognition binding moieties which bind a molecule, such that said molecule is a molecule other than a C3b-like receptor. The invention also relates to methods of producing the bispecific molecules and therapeutic and prophylactic uses thereof, as well as to kits containing the bispecific molecules.

Preferably, the bispecific molecules of the invention bind a molecule which is desired to be cleared from the circulation of a mammal, preferably a human. In a preferred embodiment, the molecule is desired to be reduced in amount in the circulation of a mammal, preferably a human. In one embodiment, the molecule is an antigen of a pathogen, i.e., a bacterium or a virus, or is a toxin. In a specific embodiment, the molecule to which the second recognition binding moiety binds is a pathogenic antigenic molecule. In another specific embodiment, the molecule is an autoimmune antigen. In yet another specific embodiment, the molecule is an antigen of an infectious disease agent. In a specific embodiment, the first recognition binding moiety binds CR1.

Any polyethylene glycol linker known in the art can be used in the methods and compositions of the invention. In a specific embodiment, the PEG linker used in the production of the bispecific molecules of the invention is a bifunctional PEG linker, having the formula, X-PEG-Y, wherein X and Y denote functional groups. In some embodiments, the X and Y functional group are the same, and hence the PEG linker is a homo-bifunctional crosslinker. In other embodiments, the X and Y functional groups are distinct. The invention encompasses derivitization of the first or second recognition binding moieties using the PEG linkers, in order to produce the bispecific molecules of the invention. The invention encompasses bispecific molecules, wherein the first or second recognition binding moieties comprise proteins, and wherein the bifunctional PEG linker derivatizes one or more amino acids within the first recognition binding moiety or the second recognition binding moieties. Any amino acid within the first or second recognition binding moiety can be derivatized using the methods of the invention. Preferably, the amino acid to be derivatized is on the surface of the first or second recognition binding moiety. In a preferred embodiment, the cross-linked bispecific molecules of the invention have the same binding activity as the first or second recognition binding moieties prior to cross-linking using a PEG linker.

The PEG linkers that can be used in the methods and compositions of the invention can be linear or non-linear molecules. Examples of non-linear PEG molecules include but are not limited to branched PEGs, linear forked PEGs, or branched forked PEGs.

The invention encompasses the use of PEG linkers, wherein the molecular weight of the PEG linker is 5 to 500 dalton. In another embodiment, the molecular weight of the PEG linkers that can be used in the methods and compositions of the invention are 200 to 20,000 dalton. In another embodiment, the molecular weight of the PEG linkers that can be used in the methods and compositions of the invention are 500 to 1000 dalton. In yet another embodiment, the molecular weight of the PEG linkers that can be used in the methods and compositions of the invention are 1000 to 8000 dalton.

The first recognition binding moiety of the bispecific molecules of the present invention can comprise any molecule that binds a C3b-like receptor (e.g. CR1). In one embodiment, the first recognition binding moiety that binds a C3b-like receptor is a an antibody that binds CR1. In a preferred embodiment, the first recognition binding moiety comprises an anti-CR1 monoclonal antibody. In one embodiment, the antibody that binds a C3b-like receptor is a monoclonal antibody, such as a murine monoclonal antibody, e.g., murine anti-CR1 antibody 7G9, a humanized monoclonal antibody, or a human monoclonal antibody. In a further specific embodiment, the antibody that binds a C3b-like receptor is a deimmunized monoclonal antibody. A deimmunized antibody refers to an antibody that is of a non-human origin but has been modified, for example with one or more amino acid substitutions so that the antibody is non-immunogenic or less immunogenic to a human when compared to the staring non-human antibody. The deimmunized antibodies for use in the methods of the invention may be made using any of the methods described in U.S. Application Serial No. 60/458,869 filed on Mar. 28, 2003 which is incorporated herein by reference in its entirety. In a specific embodiment, the deimmunized monoclonal antibody that binds CR1 is the monoclonal antibody H9, derived from the monoclonal antibody E11 (murine hybridoma E11, Catalog #184-020, Ancell Immunology Research Products, MN) which comprises of the following mutations: in the heavy chain variable region at position position 17: Ser→Thr, position 25: Thr→Ser; position 29: Ile→Met; position 44: Asn→Lys; position 45: Lys→Gly, position 49: Met→Ile; position 71: Thr→Ser; position 83: Leu→Met; and position 114: Ala→Gln; in the light chain variable region: at position 15: Leu→Val; position 53: Lys→Tyr; position 80: His→Ser; position 104: Gly→Pro; position 107: Thr→Lys; position 108: Leu→Val; and position 111: Arg→Lys.

In another embodiment, the first recognition binding moiety is a single chain Fv fragment fused to an Fc domain or a chimeric antibody having a C3b-like receptor binding domain and an Fc.

The second recognition binding moiety of the bispecific molecules of the present invention can be any molecule or a fragment thereof that binds a molecule. In particular, the molecule is desired to be cleared from the circulation of a mammal. In a preferred embodiment, the molecule is desired to be reduced in amount in the circulation of a mammal. In one embodiment, the second recognition binding moiety binds an antigenic molecule, e.g., a naturally occurring antigen of a pathogen. The antigenic molecule can be any substance that is present in the circulation of a mammal that is potentially injurious to or undesirable in a mammal, including but not limited to proteins or drugs or toxins, autoantibodies or autoantigens, or a molecule of any infectious agent or its products. The molecule to be cleared from the circulation of a mammal can be an antigenic determinant (or otherwise capable of being bound by a binding domain) that is or is part of a substance (e.g., a pathogen) that is the cause of a disease or disorder or any other undesirable condition in a mammal. The second recognition binding moiety of the invention can be any type of molecule, including but not limited to a peptide, a polypeptide, nucleic acid, oligosaccharide, or an organic small molecule.

In a preferred embodiment, the second recognition binding moiety binds the protective antigen (PA) protein of Bacillus anthracis. In yet another preferred embodiment, the second recognition binding moiety is a murine monoclonal antibody 14B7 or an antigen binding fragment thereof that binds the protective antigen (PA) protein of Bacillus anthracis.

In another embodiment, the second recognition binding moiety is an antibody or an antigen binding antibody fragment thereof that binds an antigenic molecule to be cleared from the circulation of a mammal. Antigen binding antibody fragments that can be used in the production of the bispecific molecules of the invention include but are not limited to Fab, Fab′, (Fab)′2, Fv or an sFv fragment.

In one embodiment, the bispecific molecules of the invention comprise a single second recognition binding moiety cross-linked using a PEG linker to the first recognition binding moiety. In an alternative embodiment, the bispecific molecules of the invention comprise two or more second recognition binding moieties cross-linked using a PEG linker to different regions of the first recognition binding moiety. In a specific embodiment, wherein the first recognition binding moieties comprise an antibody (i.e., an anti-CR1 antibody) and wherein the bispecific molecules of the invention contain two second recognition binding moieties, the two second recognition binding moieties may be cross-linked using a PEG linker to each of the heavy chains of the first recognition binding moiety. When two or more second recognition binding moieties are contained in the bispecific molecules of the invention, such second recognition binding moieties can be the same or different recognition binding moieties. In a preferred embodiment of the invention, the first and second recognition binding moieties target a molecule to be cleared cooperatively. In another embodiment, the first and second recognition binding moieties are different recognition binding moieties that target different molecules.

The invention encompasses a method of producing a population of bispecific molecules, said method comprising contacting an antibody that binds a C3b-like receptor with one or more recognition binding moieties, wherein said antibody is conjugated with a bifunctional poly-(ethylene)glycol (PEG) linker, and wherein said one or more recognition binding moieties are derivatized to react with the bifunctional poly-(ethylene)glycol (PEG) linker, and wherein said one or more recognition binding moieties bind a molecule; under conditions such that said derivatized recognition binding moieties react to from a covalent linkage with the PEG linker, thereby producing a population of bispecific molecules. In a specific embodiment, the molecule is desired to be cleared from the circulation of a mammal. In yet another specific embodiment, the molecule is desired to be reduced in amount in the circulation of a mammal. In a specific embodiment, derivitization of one or more recognition binding moieties comprises thiolating said one or more recognition binding moieties with a thiol specific derivatizing agent, a hydrazine or aldehyde modification agents.

The invention further encompasses a method of producing a population of bispecific molecules said method comprising:contacting an anti-CR1 antibody with NHS-poly-(ethylene)glycol (PEG)-maleimide, such that the anti-CR1 antibody is derivatized at one or more sites with the NHS functional group of the NHS-PEG-maleimide; contacting a recognition binding moiety with N-succinimidyl-S-acetyl-thioacetate (SATA), such that the recognition binding moiety is derivatized to contain one or more free thiol, and wherein said recognition binding moiety binds a molecule; combining the poly-(ethylene)glycol (PEG)-derivatized anti-CR1 antibody with the thiol derivatized recognition binding moiety; thereby producing a population of bispecific molecules. In a specific embodiment, the recognition binding moiety binds the protective antigen (PA) protein of Bacillus anthracis (Anthrax). In one embodiment, the molecule which binds the recognition binding moiety is an autoimmune antigen or an antigen of an infectious disease agent.

In a specific embodiment the invention encompasses a method of producing a population of bispecific molecules said method comprising:contacting an anti-CR1 antibody with NHS-poly-(ethylene)glycol (PEG)-benzaldehyde (PBA), such that the anti-CR1 antibody is derivatized at one or more sites; contacting a recognition binding moiety with C6 4-hydrazino-nicotinamide acetone hydrazone (Hz) such that the recognition binding moiety is derivatized, and wherein said recognition binding moiety binds a molecule; combining the poly-(ethylene)glycol (PEG)-derivatized anti-CR1 antibody with the hydrazone derivatized recognition binding moiety; thereby producing a population of bispecific molecules. The invention encompasses producing bispecific molecules using any PEG linker comprising a hydrazine/carbonyl functional group pair such as the ones disclosed and exemplified herein, e.g., NHS-poly-(ethylene)glycol (PEG)-benzaldehyde (PBA), N-hydroxy-succinimidyl-PEG-hydrazinonicotinate.

In a specific embodiment, the recognition binding moiety binds the protective antigen (PA) protein of Bacillus anthracis (Anthrax). In one embodiment, the molecule which binds the recognition binding moiety is an autoimmune antigen or an antigen of an infectious disease agent In some embodiments the invention encompasses combining the NHS-poly-(ethylene)glycol (PEG)-benzaldehyde-derivatized anti-CR1 antibody with the hydrazone derivatized recognition binding moiety; thereby producing a population of bispecific molecules.

The invention encompasses a method of producing a population of antibodies that bind a C3b-like receptor comprising a polyethylene glycol linker, said method comprising contacting the antibodies with a polyethylene glycol linker, such that the antibodies are derivatized at one or more sites with the polyethylene glycol linker, thereby producing a population of PEG-derivatized antibodies.

The invention also encompasses pharmaceutical compositions comprising a therapeutically effective amount of the bispecific molecules of the invention, said amount being effective for treating a mammal having an undesirable condition associated with the presence of said molecule in the circulation of a mammal, and a pharmaceutically acceptable carrier.

The invention encompasses kits comprising: a first container comprising a polyethylene glycol-derivatized anti-CR1 antibody; a second container comprising a recognition binding moiety, said recognition binding moiety being other than an anti-CR1 antibody; and a third container comprising a derivatizing agent suitable to derivatize said one or more recognition binding moieties.

The invention provides methods of treating a disorder in a mammal comprising administering a therapeutically effective amount of the bispecific molecules of the invention, wherein the disorder is associated with the presence of said molecule in the circulation of the mammal.

### 4. BRIEF DESCRIPTION OF THE DRAWINGS

FIG. 1 FLOW CHART SUMMARIZING AN EXEMPLARY PROCESS FOR THE CROSS-LINKING PROCEDURE FOR PRODUCTION OF 14B7IgG-PEG-7G9IgG. Illustrates schematically the steps involved in producing the bispecific molecule, 14B7IgG-PEG-7G9IgG.

FIG. 2 CHROMATOGRAPH PROFILE OF CRUDE 14B7IgG-PEG-7G9IgG. The elution profile of a crude preparation of 14B7IgG-PEG-7G9IgG is shown. The column used was Hi Prep 26/60 Sephacryl S300. The running buffer was PBSE(50 mM KPO4+150 mM NaCl+1 mM EDTA, pH 7.8.

A. This elution profile represent the profile of a crude preparation of 14B7IgG-PEG-7G9IgG as prepared using an 8:1 molar ratio; 8×NHS-PEG-maleimide: 1×7G9IgG.

B. This elution profile represent the profile of a crude preparation of 14B7IgG-PEG-7G9IgG as prepared using an 16:1 molar ratio; 16×NHS-PEG-maleimide: 1×7G9IgG.

FIG. 3 SDS-PAGE ANALYSIS OF 14B7IgG-PEG-7G9IgG The population of 14B7IgG-PEG-7G9IgG was analyzed on SDS-PAGE to determine the mobilities of each species present after SEC300 fractionation. Fractions from the HMW, LMW, and monomer fractions were analyzed.

A. Lane 1: IgM standard; Lane 2: IgA standard; Lane 3: IgG standard; Lane 4: Crude 14B7IgG-PEG-7G9IgG prepared with the 1:8 molar ratio; Lanes 5 and 6: LMW and Monomer fraction of the 1:4 molar ratio preparation; Lanes 7-9: HMW, LMW, and Monomer fractions of the 1:8 molar ratio preparation; Lanes 10-12: HMW, LMW, and Monomer fractions of the 1:16 molar ratio preparation; Lane 13: Mav 7G9 standard.

B. Lane 1: MW standard; Lanes 2-4: HMW, LMW, and Monomer fractions of the 1:4 molar ratio preparation; Lanes 5-7: HMW, LMW, and Monomer fractions of the 1:8 molar ratio preparation; Lanes 8-10: HMW, LMW, and Monomer fractions of the 1:16 molar ratio preparation

FIG. 4 MOLECULAR WEIGHT DISTRIBUTION OF 14B7-PEG-7G9 PREPARATIONS Bar graph represent the molecular weight distribution of species produced upon production of 14B7-PEG-7G9 at the 1:4, 1:8, and 1:16 molar ratios.

FIG. 5A. FLOW CHART SUMMARIZING AN EXEMPLARY PROCESS FOR THE CROSS-LINKING PROCEDURE FOR PRODUCTION OF 14B7scAb-PEG-7G9. Depicts an exemplary process for cross-linking 14B7scAb and 7G9 using SATA and NHS-PEG-MAL using 2:1 conjugation.

B. SDS-PAGE ANALYSIS OF 14B7scAb-PEG-7G9. A Tris-Glycine SDS PAGE containing the produced bispecific molecule 14B7scAb-PEG-7G9 (lanes 2 and 8).

FIG. 6A. FLOW CHART SUMMARIZING AN EXEMPLARY PROCESS FOR THE CROSS-LINKING PROCEDURE FOR PRODUCTION OF 14B7Fab-PEG-7G9. Depicts an exemplary process for cross-linking 14B7Fab and 7G9 using SATA and NHS-PEG-MAL using 2:1 conjugation.

B. SDS-PAGE ANALYSIS OF 14B7Fab-PEG-7G9. A Tris-Glycine SDS PAGE containing the produced bispecific molecule 14B7Fab-PEG-7G9 (lane 7).

FIG. 7 ELUTION PROFILE OF 14B7-HZ-PEG-H9. The Suprose6 column (Amersham) was equilibrated with PBSG ( PBS, 5% glycerol). The flow rate was 0.8 mL/min; 0.5 mL of sample was injected and fractions were collered.

FIG. 8 SDS-PAGE ANALYSIS OF 14B7-HZ-PEG-H9. The fractions from the size exclusion column were anayzed on a 3-8% tris acetate gradient gel.

### 5. DETAILED DESCRIPTION OF THE INVENTION

The present invention relates to bispecific molecules comprising a first recognition binding moiety which binds a C3b-like receptor or a functional equivalent thereof (known as complement receptor 1 (CR1) or CD35 in primates) cross-linked using a poylethylene glycol linker to one or more second recognition binding moieties which bind a molecule, such that said molecule is a molecule other than a C3b-like receptor. The invention also relates to methods of producing the bispecific molecules and therapeutic and prophylactic uses thereof, as well as to kits containing the bispecific molecules.

### 5.1 Bispecific Molecules

The present invention encompasses bispecific molecules having two or more different recognition specificities. The bispecific molecules of the invention refer to molecules comprising a first recognition binding moiety that binds a C3b-like receptor and one or more second recognition binding moieties that bind a molecule, such that said molecule is a molecule other than a C3b-like receptor. As used herein, the first recognition binding moiety comprises a chemical comprising a binding site for a C3b-like receptor, and the second recognition binding moiety comprises a chemical comprising a binding site for a molecule, e.g., a molecule to be cleared from the circulation of a mammal, such that said molecule is a molecule other than a C3b-like receptor.

In a specific embodiment, the bispecific molecules of the invention bind a molecule which is desired to be cleared from the circulation of a mammal. In another specific embodiment, the bispecific molecules of the invention bind a molecule which is desired to be reduced in amount in the circulation of a mammal. The molecule to be cleared from the circulation of a mammal can be any substance that is present in the circulation of the mammal that is potentially injurious to or undesirable in the mammal, including but not limited to proteins or drugs or toxins, autoantibodies or autoantigens, or a molecule of any infectious agent or its products. Also a molecule to be cleared from the circulation of a mammal can be a pathogenic antigenic molecule, which is any molecule containing an antigenic determinant (or otherwise capable of being bound by a binding domain) that is or is part of a substance (e.g., a pathogen) that is the cause of a disease or disorder or any other undesirable condition in a mammal. The bispecific molecules of the invention are produced by cross-linking the first and one or more second recognition binding moieties via a polyethylene glycol (PEG) linker, such that said cross-linking does not compromise the function of the first or second recognition binding moieties.

As used herein, the term “C3b-like receptor” refers to any mammalian circulatory molecule expressed on the surface of a mammalian blood cell, which has an analogous function to a primate C3b receptor, the CR1, in that it binds to a molecule associated with an immune complex, which is then chaperoned by the blood cell to, e.g., a phagocytic cell for clearance. As used herein, “epitope” refers to an antigenic determinant, i.e., a region of a molecule that provokes an immunological response in a host or is bound by an antibody. This region can but need not comprise consecutive amino acids. The term epitope is also known in the art as “antigenic determinant.” An epitope may comprise as few as three amino acids in a spatial conformation which is unique to the immune system of the host. Generally, an epitope consists of at least five such amino acids, and more usually consists of at least 8-10 such amino acids. Methods for determining the spatial conformation of such amino acids are known in the art. As used herein, an antigen-binding antibody fragment refers to a fragment of an antibody which is less than a full antibody and which comprises the antigen binding domain of the antibody.

In the present invention, the first recognition binding moiety of the bispecific molecules of the invention can be any molecule that binds a C3b-like receptor (e.g., CR1). In a specific embodiment, the first recognition binding moiety is an antibody that comprises a binding site for CR1 and an Fc domain. In a preferred embodiment, the first recognition binding moiety is an anti-CR1 antibody. In yet another preferred embodiment, the first recognition binding moiety is an anti-CR1 monoclonal antibody. In another preferred embodiment, the anti-CR1 monoclonal antibody is 7G9, HB8592, 3D9, 57F, or 1B4 (see, e.g., Talyor et al., U.S. Pat. No. 5,487,890, which is incorporated herein by reference in its entirety). In a further specific embodiment, the antibody that binds a C3b-like receptor is a deimmunized monoclonal antibody. A deimmunized antibody refers to an antibody that is of a non-human origin but has been modified, for example with one or more amino acid substitutions so that the antibody is non-immunogenic or less immunogenic to a human when compared to the starting non-human antibody. The deimmunized antibodies for use in the methods of the invention may be made using any of the methods described in U.S. Application Serial No. 60/458,869 filed on Mar. 28, 2003 which is incorporated herein by reference in its entirety. In a specific embodiment, the deimmunized monoclonal antibody that binds CR1 is the monoclonal antibody H9, derived from the monoclonal antibody E11 (murine hybridoma E11, Catalog #184-020, Ancell Immunology Research Products, MN) which comprises of the following mutations: in the heavy chain variable region at position position 17: Ser→Thr, position 25: Thr→Ser; position 29: Ile→Met; position 44: Asn→Lys; Position 45: Lys→Gly, position 49: Met→Ile; position 71: Thr→Ser; position 83: Leu→Met; and position 114: Ala→Gln; in the light chain variable region: at position 15: Leu→Val; position 53: Lys→Tyr; position 80: His→Ser; position 104: Gly→Pro; position 107: Thr→Lys; position 108: Leu→Val; and position 111: Arg→Lys.

In another embodiment, the first recognition binding moiety is an anti-CR1 antibody, including but not limited to, a single-chain variable region fragment (scFv) with specificity for a C3b-like receptor fused to the N-terminus of an immunoglobulin Fc domain.

The first recognition binding moiety can also be a chimeric antibody, such as but not limited to a humanized monoclonal antibody wherein the complementarity determining regions are mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337 which are incorporated herein by reference in their entirety). Preferably, the Fc domain of the chimeric antibody can be recognized by the Fc receptors on phagocytic cells, thereby facilitating the transfer and subsequent proteolysis of the RBC-immune complex. Although, for simplicity, this disclosure often makes references to an anti-CR1 recognition binding moiety or an anti-CR1 antibody, it is understood that such antigen recognition binding moieties or antibodies refer to any recognition binding moieties or antibodies that bind any C3b-like receptor known in the art.

In the present invention, the second recognition binding moieties of the bispecific molecules of the invention can be any molecular moiety, including but not limited to, an antibody or an antigen binding fragment thereof, that recognizes and binds a molecule to be cleared from the circulation of a mammal, e.g. a pathogenic antigenic molecule. For example, the second recognition binding moiety can be an epitope or an antigenic determinant that is bound by an antibody to be cleared from the circulatory system, such as that responsible for an autoimmune disease. The second recognition binding moiety of the bispecific molecule of the invention also encompasses a non-proteinaceous moiety. In one embodiment, the second recognition binding moiety is a nucleic acid. In another embodiment, the second recognition binding moiety is an organic small molecule. In still another embodiment, the second recognition binding moiety is an oligosaccharide.

In the present invention, the second recognition binding moiety can be an antigen binding antibody fragment of an antibody that binds an antigenic molecule. Methods for producing bispecific molecules comprising antigen binding antibody fragments are disclosed in U.S. Provisional Application No. to be assigned, Attorney docket number 9635-041-888, filed on Sep., 16 2002 which is incorporated herein by reference in its entirety. The antigen-binding antibody fragment of the bispecific molecules of the invention can be any antigen binding fragment of an antibody which recognizes and binds to a molecule to be cleared from the circulation of a mammal such as but not limited to a pathogenic antigenic molecule. Preferably, the antigen-binding antibody fragment does not comprise an Fc domain. In a preferred embodiment, the antigen-binding antibody fragment is an Fab, an Fab′, an (Fab′)₂, or an Fv fragment of an immunoglobulin molecule. Such an Fab, Fab′ or Fv fragment can be obtained, e.g., from a full antibody by enzymatic processing or from a phage display library by affinity screening and subsequent recombinant expressing (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554, each of which is incorporated herein by reference in its entirety). In another preferred embodiment, the antigen-binding antibody fragment is a single chain Fv (scFv) fragment which can be obtained, e.g., from a library of phage-displayed antibody fragments by affinity screening and subsequent recombinant expressing. In another embodiment, the antigen-binding antibody fragment is an Fab, Fab′, (Fab′)₂, Fv, or scFv fragment fused with a linker peptide of a desired length comprising a chosen amino acid sequence. In preferred embodiment, the linker peptide consists of 1, 2, 5, 10, or 20 amino acids.

In one embodiment of the invention, the bispecific molecules of the invention comprise a first recognition binding moiety (e.g. anti-CR1 monoclonal antibody) cross-linked using a poly-ethylene glycol linker to two or more second recognition binding moieties. In some embodiments, the two second recognition binding moieties are the same recognition binding moieties. In other embodiments, the two second recognition binding moieties are different recognition binding moieties. The two second recognition binding moieties can be different recognition binding moieties that target the same molecule.

In a preferred embodiment of the invention, the two second recognition binding moieties target an antigenic molecule to be cleared from the circulation of a mammal, cooperatively. As a non-limiting example, one of the second recognition binding moieties induces alterations in the conformation of the antigenic molecule so as to enhance the binding affinity of the other second recognition binding moiety, thereby facilitating the removal of the antigenic molecule from the circulation of a mammal (Thali et al., J. Acquired Immune Deficiency Syndromes 5:591-599). The two second recognition binding moieties can also be different recognition binding moieties that target different antigens to be cleared from the circulation of a mammal. The second recognition binding moieties include but are not limited to, a polypeptide, a peptide, an antigen binding domain, an epitope, a nucleic acid, or an organic small molecule.

In a preferred embodiment, the bispecific molecules of the invention comprise an anti-CR1 antibody (i.e., an anti-CR1 monoclonal antibody) cross-linked using a polyethylene glycol linker to one or more second recognition binding moieties. In a specific embodiment, the bispecific molecules of the invention comprise an anti-CR1 antibody (ie, an anti-CR1 monoclonal antibody) cross-linked using a polyethylene glycol linker to at least 1, 2, 3, 4, 5, or 6 second recognition binding moieties. Preferably, the cross-linked bispecific molecules retain the same antigenic specificity of the molecule they were derived from. In one embodiment, wherein the first recognition binding moiety comprises an antibody, the second recognition binding moiety is cross-linked using a polyethylene glycol linker at a pre-determined site on the antibody (i.e., Fc domain of an anti-CR1 antibody). Preferably, such a predetermined site does not compromise the binding of the first or the second recognition binding moiety to their respective antigens. In a most preferred embodiment, wherein the first or second recognition binding moieties comprise a protein, and wherein the first and second recognition binding moieties are cross-linked using a PEG linker at a pre-determined site, such a predetermined site is on the surface of the first or the second recognition binding moiety.

In preferred embodiments of the invention, wherein the first recognition binding moiety comprises an antibody, the second recognition binding moiety(s) is cross-linked using a polyethylene glycol linker to either the heavy or the light chain of the first recognition binding moiety (i.e., an anti-CR1 antibody). In yet another preferred embodiment, wherein the first recognition binding moiety comprises an antibody, the second recognition binding moiety(s) is cross-linked using a polyethylene glycol linker to either the heavy or the light chains of the first recognition binding moiety (i.e., an anti-CR1 antibody), with the provision that said cross-linking is not via the carboxy terminus. It is understood to one skilled in the art that other configurations are also encompassed by the invention. Non-limiting examples include but are not limited to, configurations in which one second recognition binding moiety is cross-linked using a polyethylene glycol linker to a heavy chain and another second recognition binding moiety is cross-linked using a polyethylene glycol linker to a light chain.

The invention encompasses the use of any polyethylene glycol linker known in the art for producing the bispecific molecules of the invention. The invention encompasses derivatizing the first or second recognition binding moieties of the bispecific molecules of the invention using any polyethylene glycol linker known in the art. In preferred embodiments, the polyethylene glycol linker is a bifunctional polyethylene glycol. Any method known to those skilled in the art can be used to derivatize the first or second recognition binding moieties using the polyethylene glycol linkers for use in the methods and compositions of the invention. Once the first or second recognition binding moieties have been derivatized using a polyethylene glycol linker, the other recognition domain that is to be cross-linked is derivatized or activated with any derivitization reagent known to those skilled in the art, such that it can react with the polyethylene glycol derivatized molecule to produce the cross-linked bispecific molecules of the invention. Although for simplicity, the disclosure often makes reference to the first recognition binding moiety derivatized with a polyethylene glycol linker, it will be apparent to one skilled in the art, that for producing the bispecific molecules of the invention the first or second recognition binding moieties may be derivatized with the polyethylene glycol linker and the other moiety will be derivatized with a reagent such that it will react with the polyethylene glycol derivatized moiety.

The invention also provides a polyclonal population of bispecific molecules, each comprising a first recognition binding moiety that binds a C3b-like receptor such as an anti-CR1 antibody, cross-linked using a polyethylene glycol linker with one or more second recognition binding moieties that bind a molecule. In a specific embodiment, the molecule is desired to be cleared from the circulation of a mammal. A polyclonal population of bispecific molecules of the present invention refers broadly to any population comprising a plurality of different bispecific molecules, each of which comprises an anti-CR1 antibody that binds a C3b-like receptor cross-linked via a PEG linker to a one or more other recognition binding moieties that bind a molecule. The population thus comprises a plurality of different bispecific molecules having a plurality of different binding specificities via the different recognition binding moieties. The plurality of different recognition binding moieties can recognize and bind the same epitope on a pathogen. The plurality of different recognition binding specificities can also be directed to a plurality of different epitopes on a pathogen. The plurality of different recognition binding specificities can also be directed to a plurality of variants of a pathogen. The plurality of different recognition binding specificities can further be directed to a plurality of different pathogens. The plurality of different recognition binding speciticities can further be directed to a plurality of different epitopes on a plurality of different pathogens. The characteristic and function of each bispecific molecule in the plurality of bispecific molecules in the polyclonal population can be known or unknown. The exact proportion of each bispecific molecule in the plurality of bispecific molecules in the polyclonal population can also be known or unknown. Preferably, the characteristics and the proportions of at least some bispecific molecules in the plurality of bispecific molecules in the polyclonal population are known so that if desired, the exact proportions of such members can be adjusted for optimal therapeutic and/or prophylactic efficacy. The polyclonal population of bispecific molecules can comprise bispecific molecules that do not bind the target pathogenic antigenic molecule or pathogenic antigenic molecules. For example, the population of bispecific molecules can be prepared from a hyperimmune serum that contains antibodies that bind antigenic molecules other than those that are on the target pathogens. Preferably, the plurality of bispecific molecules in the polyclonal population constitutes at least 1%, 5%, 10%, 20%, 50% or 80% of the population. More preferably, the plurality of bispecific molecules in the polyclonal population constitutes at least 90% of the population. The plurality of bispecific molecules in the polyclonal population of bispecific molecules preferably does not comprise any single bispecific molecule which has a proportion exceeding 95%, 80%, or 60% of the plurality. More preferably, the plurality of bispecific molecules in the polyclonal population of bispecific molecules does not comprise any single bispecific molecule which has a proportion exceeding 50% of the plurality. The plurality of bispecific molecules in the polyclonal population comprises at least 2 different bispecific molecules with different antigen binding specificities. Preferably, the plurality of bispecific molecules in the polyclonal population comprises at least 10 different bispecific molecules with different antigen binding specificities. More preferably, the plurality of bispecific molecules in the polyclonal population comprises at least 100 different bispecific molecules with different antigen binding specificities. The polyclonal population can be a polyclonal population generated from a suitable polyclonal population of antigen recognition portions, such as but not limited to a polyclonal immunoglobulin preparation.

### 5.2 Production of Recognition Binding Moieties

### 5.2.1 Production of Anti-CR1 Antibodies

The bispecific molecules of the invention comprise a first recognition binding moiety that binds a C3b-like receptor cross-linked using a polyethylene glycol linker to one or more second recognition binding moieties that bind a molecule. Preferably, the molecule is desired to be cleared from the circulation of a mammal. The invention encompasses derivatizing the first recognition binding moieties with any PEG linker known in the art to produce a population of PEG-derivatived molecules for use in the methods and compositions of the invention. In a preferred embodiment, the first recognition binding moiety is an antibody that binds a C3b-like receptor ( i.e., an anti-CR1 antibody). Antibodies that bind a C3b-like receptor can be derivatized at one or more sites with a PEG linker using any method known in the art. In a preferred embodiment, antibodies that bind a C3b-like receptor that are derivatized with a PEG linker have the same activity (ie., binding affinity for a C3b-like receptor) as the underivatized antibodies. In yet another preferred embodiment, antibodies that bind a C3b-like receptor that are derivatized with a PEG linker have at least 50%, 60%, 70%, 80%, 90%, 99% of the activity as the underivatized antibodies. The invention encompasses a method of producing a population of antibodies that bind a C3b-like receptor comprising a PEG linker, said method comprising contacting the antibodies with a PEG linker such that the antibodies are derivatized at one or more sites with the PEG linker, thereby producing a population of PEG-derivatized antibodies.

In a preferred embodiment, the first recognition binding moiety is an antibody that binds a C3b-like receptor. An antibody suitable for use in the present invention may be obtained from natural sources or produced by hybridoma, recombinant or chemical synthetic methods, including modification of constant region functions by genetic engineering techniques (U.S. Pat. No. 5,624,821). The antibody of the present invention may be of any isotype, but is preferably human IgG1.

In some embodiments, the anti-CR1 recognition binding moiety of the bispecific molecule comprises an anti-CR1 antibody. In preferred embodiments, the anti-CR1 recognition binding moiety of the bispecific molecule comprises an anti-CR1 mAb. An anti-CR1 mAb that binds a human C3b receptor can be produced by any method known in the art. In one embodiment, an anti-CR1 mAb, preferably an anti-CR1 IgG, can be prepared using standard hybridoma procedures known in the art (see, for example, Kohler and Milstein, 1975, Nature 256:495-497; Hogg et al., 1984, Eur. J. Immunol. 14:236-243; O'Shea et al., 1985, J. Immunol. 134:2580-2587; Schreiber, U.S. Pat. No. 4,672,044). A suitable mice is immunized with human CR1 which can be purified from human erythrocytes. The spleen cells obtained from the immunized mice are fused with an immortal mouse myeloma cell line which results in a population of hybridoma cells, including a hybridoma that produces an anti-CR1 antibody. The hybridoma which produces the anti-CR1 antibody is then selected, or ‘cloned’, from the population of hybridomas using conventional techniques such as enzyme linked immunosorbent assays (ELISA). Hybridoma cell lines expressing anti-CR1 mAb can also be obtained from various sources, for example, the murine anti-CR1 mAb that binds human CR1 described in U.S. Pat. No. 4,672,044 is available as hybridoma cell line ATCC HB 8592 from the American Type Culture Collection (ATCC). Other anti-CR1 mAbs can also be used in the present invention, see, e.g., Nickells et al., 1998, Clin. Exp. Immunol. 112:27-33. The obtained hybridoma cells are gown and washed using standard methods known in the art. Anti-CR1 antibodies are then recovered form supernatants.

In other embodiments, nucleic acids encoding the heavy and light chains of an anti-CR1 mAb, preferably an anti-CR1 IgG, are prepared from the hybridoma cell line by standard methods known in the art. As a non-limiting example, cDNAs encoding the heavy and light chains of the anti-CR1 IgG are prepared by priming mRNA using appropriate primers, followed by PCR amplification using appropriate forward and reverse primers. Any commercially available kits for cDNA synthesis can be used. The nucleic acids are used in the construction of expression vector(s). The expression vector(s) are transfected into a suitable host. Non-limiting examples include E. coli, yeast, insect cell, and mammalian systems, such as a Chinese hamster ovary cell line. Antibody production can be induced by standard method known in the art.

An anti-CR1 antibody can be prepared by immunizing a suitable subject with human CR1 which can be purified from human erythrocytes. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see, e.g., U.S. Pat. No. 5,914,112, which is incorporated herein by reference in its entirety.)

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioinmunoassay (RIA) or enzyme-linked inununo-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against human CR1 can be identified and isolated by screening a recombinant combinatorial immunoglobulin library (e.g. an antibody phage display library) with human CR1. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

An antibody can also be a single-chain antibody (scFv) which generally comprises a fusion polypeptide consisting of a variable domain of a light chain fused via a polypeptide linker to the variable domain of a heavy chain. In one embodiment, anti-CR1 scFv's are prepared according to standard methods known in the art.

In another embodiment, anti-CR1 chimeric antibodies and nucleic acids encoding such anti-CR1 chimeric antibodies are prepared according to standard methods known in the art U.S. Pat. Nos. 4,816,567, 4,816,397, 5,693,762; 5,585,089; 5,565,332 and 5,821,337 which are incorporated herein by reference in their entirety).

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, each of which is incorporated herein by reference in its entirety)

Anti-CR1 antigen recognition binding moieties can also be produced by standard phage display technologies. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

Humanized antibodies are antibody molecules from non-human species having one or more complementarity determining regions (CDRS) from the non-human species and a framework region from a human immunoglobulin molecule. (see e.g., U.S. Pat. No. 5,585,089, which is incorporated herein by reference in its entirety.) Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with human CR1. Additionally chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with human CR1.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

A pre-existing anti-CR1 antibody, including but not limited to 7G9, HB8592, 3D9, 57F, and 1B4 (see, e.g., Talyor et al., U.S. Pat. No. 5,487,890, which is incorporated herein by reference in its entirety), can also be used in the methods and compositions of the invention. In a preferred embodiment, a hybridoma cell line secreting a high-affinity anti-CR1 monoclonal antibody, e.g., 7G9 (murine IgG₂ₐ, kappa), is used to generate a master cell bank (MCB). Preferably, the master cell bank is tested for mouse antibody production, mycoplasma and sterility. The anti-CR1 antibody is then produced and purified from ascites fluid. In another preferred embodiment, the anti-CR1 monoclonal antibody used for the production of the bispecific molecules is produced in vitro (hollow-fiber bioreactor) and purified under cGMP.

### 5.2.2 Production of Recognition Binding Moeities

The invention encompasses cross-linking using polyethylene glycol linkers, a first recognition binding moiety that binds a C3b-like receptor to one or more second recognition binding moieties that bind a molecule. Preferably, the molecule is desired to be cleared from the circulation of a mammal. The recognition binding moieties of the bispecific molecules of the invention can be any molecular moiety that recognize and bind an antigenic molecule, including but not limited to an antibody or an antigen binding fragment thereof, or any molecular moiety that is recognized and bound by a molecule to be cleared, including but not limited to an epitope or an antigenic determinant, a polypeptide, a peptide, a nucleic acid, and an organic small molecule. Such recognition binding moieties can be produced by various methods known in the art.

Antibodies for use in the methods and compositions of the invention can be prepared by immunizing a suitable subject with an antigen as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g. when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Inmunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see generally Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, ie., the individual antibodies comprising the population are identical except for possible naturally occurring mutations that may be present in minor amounts. For example, the monoclonal antibodies may be made using the hybridoma method first described by Kohler et al., 1975, Nature, 256:495, or may be made by recombinant DNA methods (U.S. Pat. No. 4,816,567).

In the hybridoma method of generating monoclonal antibodies, a mouse or other appropriate host animals, such as a hamster, is immunized as described above to elicit lymphocytes that produce or are capable of producing antibodies that will specifically bind to the protein used for immunization (see U.S. Pat. No. 5,914,112, which is incorporated herein by reference in its entirety.)

Alternatively, lymphocytes may be immunized in vitro. Lymphocytes are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay(ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against a pathogen or pathogenic antigenic molecule polypeptide of the invention can be identified and isolated by screening a recombinant combinatorial immunoglobulin library (e.g., an antibody phage display library) with the antigen of interest. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734. A phage display library permits selection of desired antibody or antibodies from a very large repertoire of specificities. An additional advantage of a phage display library is that the nucleic acids encoding the selected antibodies can be obtained conveniently, thereby facilitating subsequent construction of expression vectors.

In addition, techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Nati. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323); antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Inmunol. 13:65-93). For a detailed discussion of this technology for producing human antibodies and human monoclonal antibodies and protocols for producing such antibodies, see e.g. U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif. (see, for example, U.S. Pat. No. 5,985,615)) and Medarex, Inc. (Princeton, N.J.), can be engaged to provide human antibodies directed against a selected antigen using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al. (1994) antigen Bio/technology 12:899-903).

A preexisting antibody directed against a pathogen can be used to isolate additional antigens of the pathogen by standard techniques, such as affinity chromatography or immunoprecipitation for use as immunogens. Moreover, such an antibody can be used to detect the protein (e.g., in a cellular lysate or cell supernatant) in order to evaluate the abundance and pattern of expression of the pathogen. The antibodies can also be used diagnostically to monitor pathogen levels in tissue as part of a clinical testing procedure, e.g., determine the efficacy of a given treatment regimen.

An antigenic fragment suitable for use in the methods and compositions of the invention is for example, an antigenic recognition binding moiety comprising at least a portion of the antigen that is 8 amino acids, more preferably 10 amino acids and more preferably still, 15 amino acids long. Antigens and antigenic fragments used as antigen recognition binding moieties can be recombinantly expressed or chemically synthesized.

The invention also provides chimeric or fusion antigens for use as antigen recognition binding moieties. As used herein, a “chimeric antigen” or “fusion antigen” comprises all or part of an antigen for use in the invention, operably linked to a heterologous polypeptide. Within the fusion antigen, the term “operably linked” is intended to indicate that the antigen and the heterologous polypeptide are fused in-frame to each other. The heterologous polypeptide can be fused to the N-terminus or C-terminus of the antigen.

Chimeric and fusion proteins can be produced by standard recombinant DNA techniques. In one embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (e.g., Ausubel et al., supra). Moreover, many expression vectors are commercially available that already encode a fusion domain (e.g., a GST polypeptide). A nucleic acid encoding an immunogen can be cloned into such an expression vector such that the fusion domain is linked in-frame to the polypeptide.

Other antigen recognition binding moieties of the invention can be produced using appropriate methods known in the art. For example, nucleic acids can be produced by any known method for DNA synthesis. Organic small molecules can be produced by any method know to those of skill in the art for organic synthesis.

The antigen-binding antibody fragment of the bispecific molecules of the invention can be produced by various methods known in the art.

In one embodiment, the antigen-binding antibody fragment is a fragment of an immunoglobulin molecule containing a binding domain which specifically binds a molecule to be cleared from the circulation of a mammal, e.g., pathogenic antigenic molecule. Examples of immunologically active fragments of immunoglobulin molecules include, but are not limited to, Fab, Fab′ and (Fab′)₂ fragments which can be generated by treating an antibody with an enzyme such as pepsin or papain.

In a preferred embodiment, an antigen-binding antibody fragment is produced from a monoclonal antibody having the desired antigen binding specificity. Such a monoclonal antibody can be raised using the targeted antigen by any of the standard methods known in the art. For example, a monoclonal antibody directed against an antigenic molecule can be raised using any one of the methods described, supra, using the antigenic molecule in the place of CR1 (also see section 5.2.1). The antibody is then treated with pepsin or papain. Pepsin digests an antibody below the disulfide linkages in the hinge region to produce an (Fab′)₂ fragment of the antibody which is a dimer of the Fab composed of a light chain joined to a VH-CH1 by a disulfide bond. The (Fab′)₂ fragments may be reduced under mild conditions to reduce the disulfide linkage in the hinge region thereby converting the (Fab′)₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. One of skill in the art will recognize that such Fab′ fragments may be synthesized de novo either chemically or using recombinant DNA technology. Thus, as used herein, the term antibody fragments includes antibody fragments produced by the modification of whole antibodies or those synthesized de novo.

In another embodiment, the method of generating and expressing immunologically active fragments of antibodies described in U.S. Pat. No. 5,648,237, which is incorporated herein by reference in its entirety, is used.

Methods for producing bispecific molecules comprising antigen binding antibody fragments are disclosed in U.S. Provisional Application No. to be assigned, Attorney docket number 9635-041-888, filed on Sep., 16 2002 which is incorporated herein by reference in its entirety.

In still another embodiment, the antigen-binding antibody fragment, e.g. an Fv, Fab, Fab′, or (Fab′)₂ is produced by a method comprising affinity screening of a phage display library (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554, each of which is incorporated herein by reference in its entirety). The nucleic acids encoding the antibody fragment or fragments selected from the phage display library is then obtained for construction of expression vectors. The antibody fragment or fragments can then be produced in a suitable host system, such as a bacterial, yeast, or mammalian host system (see, e.g., Plilckthun et al., Immunotechnology 3:83-105; Adair, Immunological Reviews 130:5-40; Cabilly et al., U.S. Pat. No. 4,816,567; and Carter, U.S. Pat. No. 5,648,237, each of which is incorporated herein by reference in its entirety).

In still another embodiment, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546, each of which is incorporated herein by reference in its entirety) can be adapted to produce single chain antibodies against the antigenic molecule. Single chain antibodies are formed by linking the heavy and light chain fragments of the Fv region via an amino acid bridge, resulting in a single chain polypeptide.

The invention encompasses derivatizing the recognition binding moieties that bind a molecule (i.e., the antigen-binding antibody fragment) with any derivatizing agent known in the art such that the derivatized recognition binding moiety will react with another recognition binding moiety (i.e., an anti-CR1 antibody) as discussed supra, that has been conjugated with a PEG linker. In one embodiment, the derivitization of the recognition binding moieties comprises thiolating said recognition binding moieties with a thiol specific derivatizing agent. The thiol specific derivatizing agents that can be used in the methods and compositions of the invention include but are not limited succinimidyl-3-(2-pyridylthio-propionate) (SPDP), or succinimidyl acetylthioacetate (SATA). In another embodiment, derivitization of the recognition binding moieties comprises modifying the recognition binding moieties with a hydrazine or aldehyde modification reagents. Hydrazine modification reagents or aldehyde modification reagents that can be used in the methods and the compositions of the invention are succinimidyl 6-hydrazinonicotinate acetone hydrazone (SANH) or succinimidyl 4-formyl benzoate (SFB) or succinimidyl C6 4-hydrazino-nictoinamide acetone hydrazone (Hz).

In a specific embodiment, wherein the recognition binding moieties comprise a protein, the recognition binding moieties of the invention can be modified such that they are derivatized at a predetermined site. Preferably, such a predetermined site is selected so that the binding activity of the recognition binding moiety is not compromised after derivitization or cross-linking to the anti-CR1 antibodies of the invention. Any amino acid of the recognition binding moieties may be derivatized for use in the methods and compositions of the invention, such that said derivitization does not compromise the binding of the recognition binding moiety, e.g., its binding affinity to an antigen it is directed to bind. Preferably the amino acid to be modified is a cysteine, lysine, or arginine. In one embodiment, the recognition binding moiety is derivatized at one or more sites. In a preferred embodiment, the recognition binding moiety is derivatized at only one site. In one embodiment, the recognition binding moiety is engineered using standard recombinant DNA technology to include a particular amino acid (i.e. cysteine) at a predetermined site to be derivatized. In another preferred embodiment, the amino acid to be derivatized is on the surface of the recognition binding moiety. In another embodiment, the derivatized recognition binding moiety has at least 50%, 60%, 70%, 80%, 90%, 95%, 99% of the activity of the un-derivatized recognition binding moiety.

### 5.3 Production of Bispecific Molecules Comprising a Polyethylene Glycol Linker

“Polyethylene glycol” or “PEG” refers to a polyethylene glycol compound with or without derivitization with coupling or activating moieties (e.g. with thiol, triflate, tresylate, aziridune, oxirane, or preferably maleimide). Compounds such as maleimido monomethoxy PEG are exemplary activated PEG compounds of the invention.

The present invention encompasses cross-linking the first and second recognition binding moieties of the bispecific molecules of the invention using a polyethylene glycol (“PEG”) linker, wherein such cross-linking does not destroy the binding activity of the first or second recognition binding moieties. In a specific embodiment, wherein the first recognition binding moiety is an antibody, the second recognition binding moiety(s) are preferably cross-linked via a PEG linker to the light chain or the heavy chain of the first recognition binding moiety. In yet another specific embodiment, the second recognition binding moiety(s) are cross-linked via a PEG linker to the first recognition binding moiety with the proviso, that said second recognition binding moiety is not cross-linked to the C-terminus of the first recognition binding moiety.

In a specific embodiment, the invention encompasses a method for cross-linking an anti-CR1 antibody (e.g., the 7G9 monoclonal antibody as described in U.S. Pat. No. 5,879,679) to one or more recognition binding moieties using a PEG linker. In one embodiment, the invention encompasses a method for cross-linking an anti-CR1 antibody to one or more recognition binding moieties, said method comprising contacting an anti-CR1 antibody with a PEG linker, under conditions suitable for conjugating a PEG linker to the anti-CR1 antibody, activating or derivatizing one or more second recognition binding moieties with a derivatizing agent such that it will react with the PEG linker which is conjugated to the anti-CR1 antibody, mixing the anti-CR1 antibody with the activated one or more second recognition binding moieties, under conditions suitable for cross-linking the anti-CR1 antibody to the one or more second recognition binding moieties. In another specific embodiment, the PEG linker is conjugated to the one or more second recognition binding moieties and the anti-CR1 antibody is activated or derivatized with a derivatizing agent such that it will react with the PEG linker which is conjugated to the one or more second recognition binding moieties.

Methods of conjugating a PEG linker to the first or second recognition binding moieties (i.e., antibodies, proteins) are well known in the art. Any method known in the art can be employed for the conjugation of a PEG linker for the production of the bispecific molecules of the invention. One skilled in the art can use any method known in the art to conjugate a PEG linker to anti-CR1 antibodies or other recognition binding moieties of the invention.

In specific embodiment, wherein the bispecific molecules of the invention comprise a protein, general methods of attaching a PEG linker to proteins which are disclosed for example within U.S. Pat. No. 4,179,337 issued Dec. 18, 1979, incorporated herein by reference in entirety can be used. Furthermore, other methods of attaching a PEG linker to the first or second recognition binding moieties of the invention, wherein the first or second recognition binding moieties comprise a protein can be adapted from those that are disclosed within U.S. Pat. No. 5,122,614 also incorporated herein by reference (See also Veronese et al. 1985, Applied Biochem, and Biotech, 11: 141-152; Katre et al. U.S. Pat. Nos. 4,766,106 and 4,917,888; Roberts M. J. et al., 2002 Advanced Drug Delivery Reviews, 54: 459-476; U.S. Pat. No. 5,766,897; U.S. Pat. No. 6,433,158 B1; U.S. Pat. No. 5,849,860; all of which are incorporated herein by reference in their entirety.)

In certain embodiments, the bispecific molecules of the invention comprise PEG linkers attached to at least one site, preferably at least two sites, more preferably at least three sites, most preferably at least four sites, up to a maximum number of PEG linkers, such that the attachment of PEG linkers does not abolish the binding activity of the parent first or second recognition binding moiety of the invention. The ratio of PEG linkers to any of the first or second recognition binding moieties of the invention is preferably 1:1, more preferably 2:1, even more preferably 4:1, 6:1, 8:1, up to 10:1 or 40:1 of the PEG linker to the first or second recognition binding moiety of the invention. The PEG linker attached to the first or second recognition binding moiety of the invention may range in molecular weight from 200 to 20,000 Dalton. Preferably, the PEG linker will be from 5, to 500 Dalton, 500 to 1000 Dalton or from 1000 to 8000 Dalton, more preferably from 3250 to 5000 Dalton, or about 5000 Dalton.

In a specific embodiment, wherein the bispecific molecules of the invention comprise a protein, the PEG linker are covalently attached to an amino acid residue which is on the surface of the protein and/or away from the active site.

Activated forms of PEG and monomethoxypolyethylene glycol are commercially available and may be used in the methods and compositions of the invention. Most notably, Shearwater Polymers, Inc, of Huntsville, Ala. provide a number of PEG polymers and PEG derivatives. The Shearwater Polymers Inc Catalog (Shearwaters Polymers, Inc. Catalog Functionalized Biocompatible Polymers for Research, 2001 is incorporated herein by reference and is available online at www.shearwatercorp.com) describes and make available a wide variety of activated PEGs suitable for coupling with proteins under a wide range of conditions. This catalog additionally provides preferred reaction conditions for derivatized PEG reagents. Those skilled in the art having been made aware of the numerous reagents suitable for conjugating proteins with PEG will appreciate the variety of reagent choices in view of the nature of the protein selected, the nature of the reactive amino groups or sulfhydryl groups on the protein and the end use of the conjugated protein. Activated PEGs are available which will, for example, more preferentially react with amino groups as opposed to sulfhydryl groups or vice versa Commonly selected activated PEGs include succinimidyl carbonate acitvated PEG, succinimidyl succinate PEG, and succinimidyl propionic acid PEGs. In alternative embodiments of the invention, a PEG of interest may be activated using reagents which react with hydroxyl functionalities to form a site reactive with a site on a protein of interest. In some embodiments, the protein reactive site is an amino group, a sulfhydryl group and the PEG is an active ester or imidazole (See pgs 274-185 ibid). In preferred embodiments, only one hydroxyl functionality of the PEG is activated using techniques known in the art.

In a most preferred embodiment, the invention encompasses heterofunctional PEG linkers, in which both hydroxyl groups are activated or derivatized using techniques known in the art. Heterofunctional PEG linkers have the general formula X-PEG-Y, wherein X and Y represent derivatization or functional groups (e.g., activated functional groups). A “functional group”, as used herein refers to a group of covalently attached atoms, that are either electrophillically or nucleophillically activated and can derivatize another molecule through a covalent linkage. Specific examples of functional groups include but are not limited to, COOH, —COOR, where R is lower alkyl or phenyl (carboxylic ester), —COZ, wherein Z is a halide, —CHO (aldehyde), —C(O)R (ketone), —SO2Z (wherein Z is a halide or CF3), —SO2NHZ (Z is halide), —SO2NH2, -maleimide, -amino, -alkyl halide, -alkyl-Z (where Z is mesylate, triflate or tosylate), -alkyl isocyanate, -alkyl isothiocyanate, -alkyl amine, -alkyl-OH, -alkyl-SH, -alkysulfone, -alkylsulfonamide, -alkyl aldehyde, -alkyl ketone, -alkyl-COOH, -alkyl-COOR, -alkyl-COZ (Z is halide), -alkylsulfonamide, -alkylsulfone, -alkylsulfonyl halide. All the above-mentioned functional groups may also comprise an aryl moiety rather than the alkyl moiety.

In one embodiment, wherein the bispecific molecules of the invention comprise a protein, and wherein the X and Y activated functional groups of the heterofunctional PEG linker are identical, the X and Y activated functional groups are directed to modify the same amino acid type of the first or second recognition binding moieties of the invention (e.g., an anti-CR1 antibody or a recognition binding moiety). In another embodiment, the X and Y activatied functional groups are not the same and are directed to modifying different amino acid types of the first or second recognition binding moieties of the invention (e.g., an anti-CR1 antibody or a recognition binding moiety).

In a specific embodiment, wherein the bispecific molecules of the invention comprise a protein, the amino acids of the bispecific molecules of the invention that can be modified with PEG linkers according to the methods of the invention are known in the art and include but are not limited to lysine residues (lysine residues are reactive with PEG through e-NH2), Histidine, Tryptophan, Cysteine (reactive with PEG through sulfhydryl SH; See, e.g., Goodson et al., 1990 Biotechnology 8:343), Aspartic acids (reactive with PEG through its carboxyl functionalities), Arginine, Serine (reactive with PEG through hydroxyl OH), Threonine (reactive with PEG through hydroxyl OH) or Glutamic acid (reactive with PEG through its carboxyl functionalities).

In a preferred embodiment, wherein the bispecific molecules of the invention comprise a protein, the amino acids of the first or second recognition binding moieties of the invention that are modified with PEG linkers are on the surface of the first or second recognition binding moieties. In yet another embodiment, the N-terminal amino group (See e.g., Kinstler et al., Pharm. Res. 13:1996) or the C-terminal carboxylic acid of the first or second recognition binding moieties are derivatized using PEG linkers. Conditions suitable for reaction between PEG linkers and amino acid residues within the first or second recognition binding moieties are known to those skilled in the art. Typically these procedures involve first providing an activated PEG linker in which one or both hydroxyl groups on a PEG linker are activated, and reacting the activated PEG linker with a residue within a protein selected for PEG conjugation. The general principle of PEG conjugation with proteins and common activating reagents are described in Delgado et al., 1992 in “The Uses and Properties of PEG-linked Proteins” from Critical Reviews in Therapeutic Drug Carrier Synthesis, 9(3,4):249-304 and the ACS Symposium Series 680 ed. Harries et al. Poly(ethylene glycol) Chemistry and Biological Applications 1997, both of which are incorporated herein by reference.

In some embodiments, wherein the bispecific molecules of the invention comprise a protein, the X or Y activating functional groups of the heterofunctional PEG linkers used in cross-linking the first and second recognition binding moeities of the invention are electrophillically activated by methods known in the art. At least one of the hydroxyl groups on the PEG linker is activated with a functional group (X or Y) susceptible to nucleophilic attack by the nitrogen of an amino group on a first or second recognition binding moiety of the invention. In one embodiment of the invention, electrophillically activated PEG linkers are used to modify amine residues of a first or second recognition binding moiety of the invention. The amine conjugation of PEG linkers are well known in the art, in which electrophillically activated PEG linkers target nucleophilic amine groups. Examples of PEG linkers that can be used for the modification of amine residues within a bispecific molecule of the invention include but are not limited to, PEG dichlorotriazene, PEG tresylate, PEG succinimidyl carbonate, PEG benzotriazole carbonate, PEG p-nitrophenyl carbonate, PEG trichlorophenyl carbonate, PEG carbonylimidazole, or PEG succinimidyl succinate. In preferred embodiments, electrohilically activated PEGs used in accordance of the invention are PEG succinimidyl succinate (mPEG-SS), succinimide of PEG propionic acid (mPEG-SPA), or succinimide of PEG Butanoate Acid (mPEG-SBA). Other Examples of PEG linkers that can be used for the modification of amine residues within a bispecific molecule of the invention include but are not limited to, mPEG2-H-hydroxysuccinmide (mPEG2-NHS), mPEG-Benzotriazole carbonate (mPEG-BTC), mPEG-Propionaldehyde (mPEG-ALD), mPEG-Acetaldehyde diethyl acetal (mPEG-ACET), or mPEG2-Aldehyde (mPEG2-ALD).

In a preferred embodiment, wherein the bispecific molecules of the invention comprise a protein, the X or Y activating groups of the heterofunctional PEG linkers used in producing the bispecific molecules of the invention are Lysine-active PEGs. The most preferred PEG derivative for lysine modification are N-hydroxylsuccinimide (“NHS”) active esters such as PEG succinimidyl succinate (mPEG-SS) and succinimidyl propionate (mPEG-SPA). In one embodiment, by way of example and not limitation, the following protocol is used. Equal masses of lysine-active PEG (MW, 5000) and a first or second recognition binding moiety of the invention (ie., anti-CR1 antibody) to be derivatized are mixed at pH 8-9.5, at room temperature for 30 minutes, or a time sufficient for derivatization to take place. In some embodiments, if the protein amino acid composition is known, a molar ratio of PEG (MW 5000) to protein amino groups of 1-5 to 1 is used.

In another embodiment, wherein the bispecific molecules of the invention comprise a protein, the X or Y activating functional groups of the heterofunctional PEG linkers used in producing the bispecific molecules of the invention are used for modification of cysteine residues within a bispecific molecule of the invention. Examples of PEG linkers that can be used for the modification of cysteine residues within a bispecific molecule of the invention include but are not limited to, mPEG2-forked maleimide, mPEG-forked maleimide, mPEG-maleimide, or mPEG2 maleimide. Methods for attaching PEG linkers to cysteine residues are disclosed in U.S. Pat. No. 5,766,897 which is incorporated herein by reference, in its entirety. In one embodiment, site-specific derivitization of a cysteine residue using a PEG linker can be achieved using the methods and compositions of the invention by engineering specific cysteine mutants by site-directed mutagenesis methods known in the art (Kunkel et al., 1988, Nucleic Acids and Molecular Biology, Eckstein, F. Lilley, eds., Springer-Verlag, Berling and Heidelberg, vol. 2 p.124). In yet another preferred embodiment, the bispecific molecules of the invention are cross-linked using Sulffhydryl-selective PEGs. The most preferred PEG linkers for sulfhydryl modification are vinylsulfone, iodoacetamide, and maleimide. In one embodiment, by way of example and not limitation the following protocol is used. The protein to be derivatized is mixed at pH 7-8, with a slight molar excess of PEG at room temperature for 0.5 to 2 hours.

Examples of other hetereofunctional PEG linkers that can be used in accordance with the methods and compositions of the invention include but are not limited to NHS-vinylsulfone and NHS-Maleimide (NHS-PEG-VS and NHS-PEG-Maleimide, respectively), bis-hydrazide-PEG, bis-hydrazine-PEG, and aldehyde-PEG-NHS.

In another embodiment, the heterofunctional PEG linker is a compound of Formula (I) as follows:

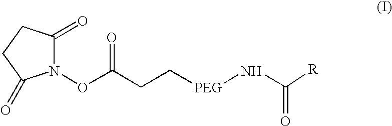

or a pharmaceutically acceptable salt therof, wherein R is phenyl, naphthyl, or aromatic heterocycle, any of which is substituted with at least one —C(O)H or —NH—NH₂ group.

“Aromatic heterocycle” refers to a 5- to 10-membered monocyclic or bicyclic aromatic carbocycle in which 1-4 of the ring carbon atoms have been independently replaced with a N, O or S atom. Representative examples of an aromatic heterocycle group include, but are not limited to, pyrrolyl, imidazolyl, benzimidazolyl, tetrazolyl, indolyl, isoquinolinyl, quinolinyl, quinazolinyl, purinyl, isoxazolyl, benzisoxazolyl, furanyl, furazanyl, pyridyl, oxazolyl, benzoxazolyl, thiazolyl, benzthiazolyl and thiophenyl.

In one embodiment, R is phenyl.

In another embodiment, R is pyridyl.

In a preferred embodiment, R is

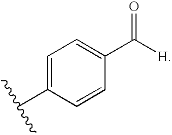

In another preferred embodiment, R is

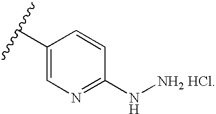

In a specific embodiment, the first recognition binding moiety that binds a C3b-like receptor (i.e., an anti-CR1 antibody, e.g., an anti-CR1 monoclonal antibody) is derivatized with NHS-PEG-maleimide. By way of example, and not limitation, the protocol for NHS-PEG-maleimide can be as follows: The anti-CR1 antibody is derivatized with NHS-PEG-maleimide at a molar ratio of 6:1; 6×NHS-PEG-maleimide: 1× anti-CR antibody, such that the reaction proceeds at room temperature for two hours at gentle inversion every 15-30 minutes, wherein the anti-CR1 antibody is derivatized at one or more sites with NHS-PEG-maleimide. The resulting product from the derivitization is then desalted by chromatography using standard procedures known in the art (e.g. using an Amersham Hi-Prep 26/10 desalting column in MES buffer).

In yet another specific embodiment, the first recognition binding moiety that binds a C3b-like receptor (i.e., an anti-CR1 antibody, e.g., an anti-CR1 monoclonal antibody) is derivatized with NHS-PEG-benzaldehyde. Modification using NHS-PEG-benzaldehyde may have several advantages relative to other modification procedures such as those involving maleimide chemistry. Although not intending to be bound by a particular mechanism of action, molecules, e.g., antibodies, modified with NHS-PEG-benzaldehyde tend to be stable over an extended period of time, e.g. at least one month, because the hydrazone or aldehyde moiety is stable under the pH range where the antibody is typically stored. Therefore, the antibody derivatization reaction can be carried out well in advance of the conjugation reaction. Modification using NHS-PEG-benzaldehyde may thus be preferred for commercial production, because the production schedule can be more flexible and the unconjugated monomeric fraction can be recycled. Another benefit of modifying antibodies with NHS-PEG-benzaldehyde is that the hydrazine or aldehyde chemistry will not lead to bond formation with other functional groups in the antibody; any weak bond that could form between the amino group and the aldehyde is hydrolyzed in the aqueous buffer under physiological conditions. When modifying antibodies using maleimide chemistry, however, the derivatized antibodies might react with the free sulfhydryl group on the antibody, leading to an undesired modification. Yet another particular benefit of the NHS-PEG-benzaldehyde linker of the invention is that it requires no reducing agent for a stable bond formation over the pH range where antibodies are typically maintained in the stable form. While sulflhydryl modified proteins may form homodimers, there is no homodimer formation of the antibody using the hydrazone linker. Yet another benefit of using the hydrazine chemistry is that the reaction kinetics of hydrazine/carbonyl linkage is fast and can be carried out in a condition where the antibody can be maintained in the active form.

The invention encompasses derivatizing the first or second recognition binding moieties of the invention using PEG linkers using any protocol known to those skilled in the art. It will be apparent to one skilled in the art, that the molar ratio of the PEG linker used in derivatizing the first or second recognition binding moieties of the invention, will depend on the molecular weight of the PEG linker used and the molecular weight of the molecule being derivatized. One skilled in the art can determine the molar ratio of the PEG linker to be used in the derivitization of the first or second recognition binding moieties using routine experimentation. In a specific embodiment, for derivitazation of NHS-PEG-maleimide to the first or second recognition binding moieties of the invention the molar ratio of the NHS-PEG-maleimide to the first or second recognition binding moieties is 3:1, 4:1, 5:1, 6:1, or 8:1.

Linear PEG linkers are the most preferred cross-linking reagents in accordance with the invention. In some embodiments, other cross-linking reagents are encompassed by the invention. Examples of additional cross-linking reagents include but are not limited to, modified PEG linkers, branched PEG linkers (e.g., PEG2), linear forked PEG linkers, branched forked PEG linkers, or cross-linked PEG linkers.

In some embodiments, cross-linking the first and second recognition binding moieties of the bispecific molecules of the invention using PEG linkers are done in a site-directed manner. In a specific embodiment, wherein the first or second recognition binding moiety of the invention comprises an antibody. a PEG linker is conjugated site-specifically to oxidized carbohydrate residues within the Fc region of the first or second recognition binding moieties. Methods to oxidize carbohydrates are well known in the art, and include but are not limited to enzymatic oxidation (e.g. glucose oxidase) or chemical oxidation (e.g., periodate). Oxidation of carbohydrate residues generates multiple reactive aldehyde groups which can be conjugated with PEG linkers that have for example, an amine or a hydrazide functional group.

The invention encompasses methods of cross-linking a first and second recognition binding moiety using, heterofunctional PEG linkers, having the formula, X-PEG-Y. Once a first recognition binding moiety has been derivatized with a heterofunctional PEG linker (e.g., using the X-functional group), the resulting PEG derivatized recognition binding moiety will be combined at a desired molar ratio, with an activated or derivatized second recognition binding moiety, such that the second activated or derivatized recognition binding moiety will react with a functionality of the PEG linker that is free to react on the first PEG-derivatized recognition binding moiety. A skilled person in the art will be able to determine the molar ratio of the PEG-derivatized first recognition binding moiety and the derivatized second recognition binding moiety. In a specific embodiment, the first recognition binding moiety is anti-CR1 antibody.

Techniques of activating or derivatizing the first or second recognition binding moieties are well known in the art and any method known in the art can be used in accordance with the invention. Recognition binding moieties for example, can be thiolated using reagents and methods known in the art, in order to react with PEG derivatives directed at sulfhydryl groups. For examples, amines of recognition binding moieties of the invention can be indirectly thiolated by reaction with succinimidyl 3-(2-pyridyldithio)propionate (“SPDP”), followed by reduction with DTT or tris-(2-carboxyethyl)phospohine (“TCEP”). Amines can also be thiolated by reaction with succinimidyl acetylthioacetate (“SATA”) followed by removal of the acetyl group with 50 mM hydroxylamine or hydrazine at or near neutral pH. Additionally, thiols can be incorporated at carboxylic acid groups by an EDAC mediated reaction with cystamine followed by reduction of the disulfide with DTT or TCEP. Other techniques for thiolation of the first or second recognition binding moieties are well known in the art and can be used in the methods of the invention.

In a specific embodiment, the invention encompasses cross-linking using PEG cross linkers a first recognition binding moiety to a second recognition binding moiety that binds the protective antigen (PA) protein of Bacillus anthracis (Anthrax). In yet another specific embodiment the second recognition binding moiety that binds the protective antigen (PA) protein of Bacillus anthracis (Anthrax) is a murine monoclonal antibody 14B7. In a specific embodiment 14B7 is derivatized with SATA at one or more sites in order to react with a PEG-derivatized first recognition binding moiety that has been derivatized according to the methods of the invention. By way of example and not by limitation 14B7 is derivatized with SATA using the following protocol: 14B7 is dialysed in PBSE buffer overnight at 4C; SATA is reacted with the dialyzed 14B7 at a molar ratio of 6:1 (6×SATA: 1×14B7) at room temperature for two hours with gentle inversion every 15-30 minutes. Hydroxylamine hydrochloride at a molar ratio of 2000:1 (2000×hydroxylamine hydrochloride:1×SATA-derivatized 14B7 ) is then added to the reaction mixture and the mixture is reacted at room temperature for two hours under Argon gas. The mixture is subsequently desalted using standard procedures known to those skilled in the art (i.e., Amersham Hi-Prep desalting column (26/10) in MES buffer).

Recognition binding moieties of the invention can be modified using hydrazine or aldehyde amine modification reagents for example with, “SANH”; succinimidyl 6-hydrazinonicotinate acetone hydrazone or “SFB”; succinimydyl 4-formylbenzoate.

Various methods known in the art optionally can be used to assess the derivitization of PEG linkers with of the first or second recognition binding moieties of the invention. One skilled in the art can use assays to determine the number of PEG linkers attached to a first or second recognition binding moiety of the invention, and the different PEG-derivatized moieties formed as a result of the derivitization of the PEG linkers to the first or second recognition binding moieties of the invention.

In a specific embodiment, where the bispecific molecules of the invention comprise a protein, the specific amino acids that have been modified with a PEG linker can be determined. In one specific embodiment, where a lysine residue of a first or second recognition binding moiety of the invention has been derivatized with a PEG linker, unmodified lysine groups can be determined using the “Habeeb Method” wherein unmodified lysine groups react with trinitrobenzenesulfonic acid followed by UV measurement (Habeeb, 1966 Anal Biochem. 14:328; Karr et al., 1986, J. Chrom. 354:269; Abuchowski et al., 1977 J. Biol. Chem. 252:3578). Another method for determining the unmodified lysine groups is the fluorescamine method of Stocks in which fluorescamine is reacted with unmodified lysine groups yielding a fluorescent derivative (Karr et al. 1994, Methods in Enzymology, 228: 377). In another embodiment, where a cysteine residue of a first or second recognition binding moiety of the invention has been derivatized with a PEG linker, available cysteine groups can be determined by a spectrophotometric assay based on reaction with 2,2′-dipyridyl disulfide which forms 2-thiopyridone, which absorbs at 343 nm with e=7060 at pH 7.2. Another approach is reaction with Ellman's reagent, 5,5′-dithiobis(2-nitrobenzoic acids) (See Grassetti et al., 1967 Biochem. Biophys., 119:41; Riddles et al., 1979, Anal. Bioch, 94:75).

### 5.3.1 Purification and Characterization of the Bispecific Molecules

The population of the bispecific molecules produced by the methods of the invention such as described supra are preferably purified. Bispecific molecules can be purified by any method known to one skilled in the art using purification techniques comprising molecular size exclusion of the population of the bispecific molecules or specific binding affinity of the population of the bispecific molecules or a combination thereof.

The invention encompasses purifying the population of the bispecific molecules produced by the methods of the invention by ion exchange chromatography using columns suitable for isolation of the bispecific molecules of the invention including DEAE, Hydroxylapatite, Calcium Phosphate (see generally Current Protocols in Immunology, 1994, John Wiley & Sons, Inc., New York, N.Y.).

In another embodiment, the population of the bispecific molecules produced by the methods of the invention are purified by three-step successive affinity chromatography (Corvalan and Smith, 1987, Cancer Immunol. Immunother., 24:127-132): the first column is made of protein A bound to a solid matrix, wherein the Fc portion of the antibody binds protein A, and wherein the antibodies bind the column; followed by a second column that utilizes C3b-like receptor bound to a solid matrix which assays for C3b-like receptor binding via the anti-CR1 mAb portion of the bispecific molecule; and followed by a third column that utilizes specific binding of an antigenic molecule of interest which binds the antigen recognition binding moiety of the bispecific molecule.

The invention also encompasses purifying the population of the bispecific molecules produced by the methods of the invention, by a combination of size exclusion chromatography, high performance liquid chromatography (HPLC) and affinity chromatography. In one embodiment, the appropriate fraction eluted from of size exclusion chromatography, high performance liquid chromatography (HPLC) is further purified using a column containing an antigenic molecule specific to the antigen recognition binding moiety of the bispecific molecule.

The invention further encompasses preferably characterizing the bispecific molecules of the invention using any method known in the art. The yield of the bispecific molecules of the invention can be characterized based on the protein concentration. In one embodiment, the protein concentration is determined using a Lowry assay. Preferably, the bispecific molecules produced by the method of the present invention has a protein concentration of at least 0.100 mg/ml, more preferably at least 0.5 mg/ml, still more preferably at least 2.0 mg/ml, most preferably at least 10 mg/ml. In another embodiment, the concentration of the bispecific molecules of the invention is determined by measuring UV absorbance spectroscopy. The concentration is determined by measuring the absorbance of the bispecific molecules at 280 nm. Preferably, the bispecific molecules produced by the method of the present invention have an absorbance at 280 nm of at least 0.14.

The bispecific molecules of the invention can also be characterized using any other standard method known in the art. In one embodiment, high-performance size exclusion chromatography (HPLC-SEC) assay is used to determined the content of contamination by free IgG proteins. In preferred embodiments, the bispecific molecule composition produced by the method of the present invention has a contaminated IgG concentration of less than 50%, more preferably less than 30%, most preferably less than 10%.

In one embodiment, the bispecific molecules of the invention can be characterized by using SDS-PAGE to determine the molecular weight of the species in the population of the bispecific molecules produced by the methods of the invention.

In a preferred embodiment, the invention encompasses a homogenous population of the bispecific molecules produced by the methods of the invention, wherein at least 90% of the species of the bispecific molecules in the population is a dimeric cross-linked species, as determined by standard methods in the art (i.e., mobility on SDS-PAGE; elution profile on size exclusion chromatography). In another embodiment, the invention encompasses a homogenous population of the bispecific molecules produced by the methods of the invention, wherein at least 50%, 60%, 70%, or 80% of the species of the bispecific molecules in the population is a dimeric cross-linked species, as determined by standard methods in the art (i.e., mobility on SDS-PAGE; elution profile on size exclusion chromatography)

The invention further encompasses characterizing the bispecific molecules of the invention based on the functional activity of the bispecific molecules. In one embodiment, the anti-CR1 binding activity is determined using ELISA with immobilized CR1 receptor molecules (attached to a solid phase, e.g., a microtiter plate) (see Porter et al., U.S. provisional application No. 60/380,211, which is incorporated herein by reference in its entirety). The assay is also referred to as a CR1/Antibody assay or CAA, and can be used generally to measure any anti-CR1 antibody, or HP or AHP containing an anti-CR1 antibody. In a preferred embodiment, ELISA/CR1 plates are prepared by incubating ELISA plates, e.g., high binding flat bottom ELISA plates (Costar EIA/RIA strip plate 2592) with a suitable amount of a bicarbonate solution of CR1 receptors. Preferably, the concentration of the bicarbonate solution of CR1 receptors is 0.2 ug/ml prepared from 5 mg/ml sCR1 receptors stock (Avant Technology Inc.) and a carbonate-bicarbonate buffer (pH 9.6, Sigma C-3041). In a preferred embodiment, 100 ul CR1-bicarbonate solution is dispensed into each well of the ELISA plates and the plates are incubated at 4° C. overnight. The plates are then preferably washed using, e.g. a wash buffer (PBS, 0.1% Tween-20, 0.05% 2-Chloroacetamide). In another preferred embodiment, a SuperBlock Blocking Buffer in PBS (Pierce) is added to the plates for about 30-60 min at room temperature after the wash. The plates can then be dried and stored at 4° C. The titration of anti-CR1 Abs or bispecific molecules can be carried out using a CR1 binding protein, e.g., human anti-CR1 IgG, as the calibrator. In a preferred embodiment, the calibrator a human anti-CR1 IgG having a concentration of 300 or 600 mg/ml. In one embodiment, the titration of the purified composition of bispecific molecules of the invention is carried out using PBS, 0.25% BSA, 0.1% Tween-20 as the diluent buffer, PBS, 0.1% Tween-20, 0.05% 2-Chloroacetamide as the wash buffer, TMB-Liquid Substrate System for ELISA (3,3′,5.5′-Tetramethyl-Benzidine) and 2N H₂SO₄ as the stop solution. Preferably, the bispecific molecule produced by the method of the present invention has an CAA titer of at least 0.03 mg/ml, more preferably at least 2.0 mg/ml, and most preferably at least 6.0 mg/ml. In some embodiments, a specific anti-CR1 activity is determined. The specific anti-CR1 activity is a ratio of CAA and Lowry.

The antigen-binding activity of the bispecific molecules of the invention can be determined using ELISA with immobilized antigen molecules.

In another embodiment, the bispecific molecule comprising an antibody that binds a C3b-like receptor cross-linked with an antigen-binding antibody fragment that binds the protective antigen (PA) protein of Anthrax is characterizied for its binding of the PA antigen using an ELISA assay. The assay is also referred to as an HPCA assay. The HPCA assay is used to analyze the functionality of the cross-linked bispecific molecules of the invention in terms of the binding specificity of the bispecific molecules to CR-1 and PA.

By way of example, and not limitation the following protocol can be used. Plates (Corning Costar Assay plate, v-bottom non-treated polystyrene) are coated with CR-1 at a concentration of 0.2 μg/ml. An anti-PA heteropolymers is used as an internal standard, 14B7x7G9 at a concentration of 464.0 μg/ml. Various control concentrations were used High Control (“HC”) 1.0 μg/ml, Medium Control (“MC) 0.5 μg/ml, Low Control (“LC”) 0.25 μg/ml. Biotin conjugated PA is used at a concentration of 0.81 mg/ml. The ELISA Diluent Buffer contains 1×PBX buffer, 0.25% BSA, 0.1% Tween 20,0.05% 2-Chloroacetamide. The ELISA Wash Buffer contains 1×PBS, 0.1% Tween-20, 0.05% 2-Chloroacetamide. 3,3′, 5.5′-tetramethyl-benzedene (“TMB”) is obtained from Sigma (cat #T-0440, LOT #21K1392). The stop solution contains 2N H₂SO₄. Horse radish Peroxidase-conjugated Streptavidin; SA-HRP is provided at 0.5 mg/ml.

Initially the antibody that binds a C3b-like receptor (i.e., anti-CR1 antibody (7G9)) which is PEG-cross-linked to an antibody or an antigen binding fragment thereof that binds PA (ie., 14B7) is bound to the CR-1 plate using the following procedure. The PEG-crosslinked bispecific antibody is diluted to 5 μg/ml in the ELISA diluent buffer. In a dilution plate, samples are loaded at 5 μg/ml in rows A through H and serially diluted 1:3 fold.

100 μl of diluted samples are transferred from the dilution plate into corresponding wells on the CR-1 coated plate. 100 μl of HC, MC, and LC are added in duplicates to rows A11 and A12, B11 and B12, C11 and C12, respectively. 100 μl of diluent are added for blanks to five wells in duplicates. The plate is then sealed with the adhesive plate sealer and incubated at 37° C. for 1 hour. The solution is discarded and the plate is washed on auto plate washer with 5-cycle program.

Next biotinylated PA (“b-PA”) is bound to the PEG-crosslinked bispecific antibody using the following procedure. b-PA is diluted to 2.5 ng/ml in ELISA diluent buffer. 100 μl of diluted b-PA is transferred into all wells (including blank wells). The plate is then sealed with the adhesive plate sealer and incubated at 37° C. for 1 hour. The solution is discarded and the plate is washed on auto plate washer with 5-cycle program.

Finally streptavidin conjugated horseradish peroxidase (“SA-HRP”) is bound to b-PA using the following method. SA-HRP is diluted 1:10,000 in ELISA diluent buffer.

100 μl of diluted SA-HRP is transferred into all wells (including blank wells). The plate is then sealed with the adhesive plate sealer and incubated at 37° C. for 1 hour. The solution is discarded and the plate is washed on auto plate washer with 5-cycle program.

In order to develop signal, 100 μl of pre-warmed TMB is added to all wells. The plate is incubated at room temperature for 15 min (protected from light). 100 μl of stop solution (2N H₂SO₄) is addend, and the plate is additionally incubated at room temperature for another 10 min. The plate is read at 450 nm using a plate reader.

The maximal absorbance value obtained, referred to as Max OD, can be used as a measure of the total activity of the bispecific molecule. In a preferred embodiment, Max OD is obtained from a 4-parameter sigmoidal fit of the optical density data In another embodiment, a C₅₀ level is also determined. The C₅₀ is the concentration of a sample which yields 50% of the max OD.

### 5.4 Uses of Bispecific Molecules

The bispecific molecules of the present invention are useful in treating or preventing a disease or disorder associated with the presence of a pathogenic antigenic molecule. The pathogenic antigenic molecule can be any substance that is present in the circulation that is potentially injurious to or undesirable in the subject to be treated, including but not limited to proteins or drugs or toxins, autoantibodies or autoantigens, or a molecule of any infectious agent or its products. A pathogenic antigenic molecule is any molecule containing an antigenic determinant (or otherwise capable of being bound by a binding domain) that is or is part of a substance (e.g., a pathogen) that is the cause of a disease or disorder or any other undesirable condition.

The preferred subject for administration of a bispecific antibody of the invention, for therapeutic or prophylactic purposes, is a mammal including but not limited to non-human animals (e.g., horses, cows, pigs, dogs, cats, sheep, goats, mice, rats, etc.), and in a preferred embodiment, is a human or non-human primate.

Circulating pathogenic antigenic molecules cleared by the fixed tissue phagocytes include any antigenic moiety that is harmful to the subject. Examples of harmful pathogenic antigenic molecules include any pathogenic antigenic molecule associated with a parasite, fungus, protozoa, bacteria, or virus. Furthermore, circulating pathogenic antigenic molecules may also include toxins, immune complexes, autoantibodies, drugs, an overdose of a substance, such as a barbiturate, or anything that is present in the circulation and is undesirable or detrimental to the health of the host mammal. Failure of the immune system to effectively remove the pathogenic antigenic molecules from the mammalian circulation can lead to traumatic and hypovolemic shock (Altura and Hershey, 1968, Am. J. Physiol. 215:1414-9).

Moreover, non-pathogenic antigens, for example transplantation antigens, are mistakenly perceived to be harmful to the host and are attacked by the host immune system as if they were pathogenic antigenic molecules. The present invention further provides an embodiment for treating transplantation rejection comprising administering to a subject an effective amount of a bispecific molecule of the invention that will bind and remove immune cells or factors involved in transplantation rejection, e.g., transplantation antigen specific antibodies.

### 5.4.1 Autoimmune Antigens

In one embodiment, the pathogenic antigenic molecule to be cleared from the circulation includes autoimmune antigens. These antigens include but are not limited to autoantibodies or naturally occurring molecules associated with autoimmune diseases.

As one example, certain humans with hemophilia have been shown to be deficient in factor VIII. Recombinant factor VIII replacement treats this hemophilia. However, eventually some patients develop antibodies against factor VIII, thus interfering with the therapy. The bispecific antibodies of the present invention prepared with an anti-anti-factor VIII antibody provide a therapeutic solution for this problem. In particular, a bispecific antibody with specificity of the first recognition binding moiety to a C3b-like receptor and specificity of the second recognition binding moiety to an anti-factor VIII autoantibody would be therapeutically useful in clearing the autoantibodies from the circulation, thus, ameliorating the disease.

Further examples of autoantibodies which can be cleared by the bispecific antibodies of the present invention include, but are not limited to, autoantibodies to the following antigens: the muscle acetylcholine receptor (the antibodies are associated with the disease myasthenia gravis); cardiolipin (associated with the disease lupus); platelet associated proteins (associated with the disease idiopathic thrombocytopenic purpurea); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is characterized and is associated with disease pathogenesis.

When the above bispecific antibodies are injected into the circulation of a human or non-human primate, the bispecific antibodies will bind to red blood cells via the human or primate C3b receptor domain recognition site, at a high percentage and in agreement with the number of C3b-like receptor sites on red blood cells. The bispecific antibodies will simultaneously associate with the autoantibody indirectly, through the antigen, which is bound to the monoclonal antibody. The red blood cells which have the bispecific antibody/autoantibody complex on their surface then facilitate the neutralization and clearance from the circulation of the bound pathogenic autoantibody.

In the present invention, the bispecific antibodies facilitate pathogenic antigen or autoantibody binding to hematopoietic cells expressing a C3b-like receptor on their surface and subsequently clear the pathogenic antigen or autoantibody from the circulation, without also clearing the hematopoietic cells.

### 5.4.2 Infectious Diseases

In specific embodiments, infectious diseases are treated or prevented by administration of a bispecific molecule of the invention that binds both an antigen of an infectious disease agent and a C3b-like receptor. Thus, in such an embodiment, the pathogenic antigenic molecule is an antigen of an infectious disease agent.

Such antigens include but are not limited to: influenza virus hemagglutinin (Genbank accession no. JO2132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), core protein, matrix protein or other protein of Dengue virus (Genbank accession no. M19197; Hahn et al., 1988, Virology 162:167-180), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Seipulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales-Scarano et al., 1982, Virology 120:42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published June 4, 1980; Ganem and Varmus, 1987, Ann. Rev. Biochem. 56:651-693; Tiollais et al., 1985, Nature 317:489-495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

Additional diseases or disorders that can be treated or prevented by the use of a bispecific molecule of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picomaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus 1 (B virus), and poxviruses.

Bacterial diseases or disorders that can be treated or prevented by the use of bispecific molecules of the present invention include, but are not limited to, Mycobacteria rickettsia, Mycoplasma, Neisseria spp. (e.g., Neisseria menigitidis and Neisseria gonorrhoeae), Legionella, Vibrio cholerae, Streptococci, such as Streptococcus pneumoniae, Corynebacteria diphtheriae, Clostridium tetani, Bordetella pertussis, Haemophilus spp. (e.g., influenzae), Chlamydia spp., enterotoxigenic Escherichia coli, and Bacillus anthracis (anthrax), etc.

Protozoal diseases or disorders that can be treated or prevented by the use of bispecific molecules of the present invention include, but are not limited to, plasmodia, eimeria, Leishmania, and trypanosoma.

In a specific embodiment, the invention provides a method and compositions for treating Anthrax infection. The method comprises administering to a patient a therapeutically effective amount of a bispecific molecule comprising an antibody that binds a C3b-like receptor cross-linked using a PEG linker with a full length antibody (i.e., 14B7 murine monoclonal antibody) or an antigen binding fragment thereof which binds the protective antigen (PA) protein of Bacillus anthracis (Anthrax), a common component of the lethal and edema toxins of Anthrax (see, e.g., Little et al., 1991, Biochem Biophys Res Commun.180:531-7; Little et al., 1988, Infect Immun. 56:1807-13). The protective antigen protein of Anthrax was shown to be required for toxicity (Little et al., 1988, Infect Immun. 56:1807-13). The bispecific molecules can be used to remove PA from the circulation thereby ameliorating the toxic effect of Anthrax. Methods for producing bispecific molecules comprising antigen binding antibody fragments of an antibody that binds the PA protein are disclosed in U.S. Provisional Application No. to be assigned, Attorney docket number 9635-041-888, filed on Sep. 16, 2002 which is incorporated herein by reference in its entirety.

In one embodiment, the antibody fragment is the Fab fragment of an antibody 14B7 which binds PA (see, e.g., Little et al., 1991, Biochem Biophys Res Commun.180:531-7; Little et al., 1988, Infect Immun. 56:1807-13). In another embodiment, the antibody fragment is a single-chain antibody derived from 14B7 (14B7scAb). The 14B7scAb consists of a single chain Fv of 14B7 fused with a human constant k domain (see, e.g., Maynard et al., Nature Biotechnology 20:597-601). In a preferred embodiment, the antibody that binds a C3b-like receptor is the murine anti-CR1 IgG 7G9. In a preferred embodiment, the bispecific molecule is produced by cross-linking an anti-CR1 mAb, e.g., 7G9, and an anti-PA Fab fragment, e.g. 14B7Fab, using N-succinimidyl-S-acetyl-thioacetate (SATA) and sulfosuccinimidyl 4-(N-maleimidomethyl) cyclohexane-1-carboxylate (sSMCC) as the cross-linking agents. In another preferred embodiment, the bispecific molecule is produced by cross-linking an anti-CR1 mAb, e.g., 7G9, and an anti-PA single chain antibody, e.g., 14B7scAb, using N-succinimidyl-S-acetyl-thioacetate (SATA) and NHS-poly(ethylene glycol)-maleimide (PEG-MAL) as the cross-linking agents. In still another preferred embodiment, the bispecific molecule is produced by cross-linking an anti-CR1 mAb, e.g., 7G9, and an anti-PA single chain antibody, e.g., 14B7Fab, using N-succinimidyl-S-acetyl-thioacetate (SATA) and NHS-poly(ethylene glycol)-maleimide (PEG-MAL) as the cross-linking agents.

### 5.4.3 Addictional Pathogenic Antigens

In one embodiment, the pathogenic antigenic molecule to be cleared from the circulation by the methods and compositions of the present invention encompass any serum drug, including but not limited to barbiturates, tricyclic antidepressants, and Digitalis.

In another embodiment, the pathogenic antigenic molecule to be cleared includes any serum antigen that is present as an overdose and can result in temporary or permanent impairment or harm to the subject. This embodiment particularly relates to drug overdoses.

In another embodiment, the pathogenic antigenic molecule to be cleared from the circulation include naturally occurring substances. Examples of naturally occurring pathogenic antigenic molecules that could be removed by the methods and compositions of the present invention include but are not limited to low density lipoproteins, interleukins or other immune modulating chemicals and hormones.

### 5.4.4 Cocktails of Bispecific Molecules

Various purified bispecific molecules of the invention can be combined into a “cocktail” of bispecific molecules. Such cocktail of bispecific molecules can include bispecific molecules having an anti-CR1 mAb as the first recognition binding moiety and any one of several desired recognition binding moiety as the second recognition bindingmoieties. For example, the bispecific molecule cocktail comprises a plurality of different bispecific molecules, wherein each different bispecific molecule in the plurality contains a different second recognition binding moiety that targets a different pathogen; the second recognition binding moiety can be proteinaceous and/or non-proteinaceous moieties. Such bispecific molecule cocktails are useful as personalized medicine tailored according to the need of individual patients.

### 5.5 Pharmaceutical Compositions and Administration

The bispecific molecules of the invention can be incorporated into pharmaceutical compositions suitable for administration to a mammal, preferably a human. Such compositions typically comprise bispecific molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the bispecific antibody, use thereof in the compositions is contemplated. Supplementary bispecific antibodies can also be incorporated into the compositions.

A pharmaceutical composition of the invention is formulated to be compatible with its intended route of administration. The preferred route of administration is intravenous. Other examples of routes of administration include parenteral, intradermal, subcutaneous, transdermal (topical), and transmucosal. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that the viscosity is low and the bispecific antibody is injectable. It must be stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the bispecific molecule (e.g., one or more bispecific antibodies) in the required amount in an appropriate solvent with one or a combination of ingredients enumerated above, as required, followed by filtered sterilization. Generally, dispersions are prepared by incorporating the bispecific molecule into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the bispecific molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference in its entirety.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of bispecific antibody calculated to produce the desired therapeutic effect in association with the required pharmaceutical carrier. The specification for the dosage unit forms of the invention are dictated by and directly dependent on the unique characteristics of the bispecific antibody and the particular therapeutic effect to be achieved, and the limitations inherent in the art of compounding such a bispecific antibody for the treatment of individuals.

The pharmaceutical compositions can be included in a kit, in a container, pack, or dispenser together with instructions for administration.

### 5.5.1 Doses of Bispecific Antibodies

The dose of a bispecific molecule of the invention can be determined by a physician upon conducting routine experiments. Prior to administration to humans, the efficacy is preferably shown in animal models. Any animal model for a circulatory disease known in the art can be used.

More particularly, the dose of a bispecific antibody can be determined based on the hematopoietic cell concentration and the number of C3b-like receptor epitope sites bound by the anti-C3b-like receptor monoclonal antibodies per hematopoietic cell. If the bispecific antibody is added in excess, a fraction of the bispecific antibody will not bind to hematopoietic cells, and will inhibit the binding of pathogenic antigens to the hematopoietic cell. The reason is that when the free bispecific antibody is in solution, it will compete for available pathogenic antigen with bispecific antibody bound to hematopoietic cells. Thus, the bispecific antibody-mediated binding of the pathogenic antigens to hematopoietic cells follows a bell-shaped curve when binding is examined as a function of the concentration of the input bispecific antibody concentration.

In general, for antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

As defined herein, a therapeutically effective amount of a bispecific antibody (ie., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but is not limited to the severity of the disease or disorder, previous treatments, the general health and/or age of the subject, and other diseases present. Moreover, treatment of a subject with a therapeutically effective amount of a bispecific antibody can include a single treatment or, preferably, can include a series of treatments. In a preferred example, a subject is treated with a bispecific antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a bispecific antibody, used for treatment may increase or decrease over the course of a particular treatment. Changes in dosage may result and become apparent from the results of diagnostic assays as described herein.

It is understood that appropriate doses of bispecific antibody agents depends upon a number of factors within the ken of the ordinarily skilled physician, veterinarian, or researcher. The dose(s) of the bispecific antibody will vary, for example, depending upon the identity, size, and condition of the subject or sample being treated, further depending upon the route by which the composition is to be administered, if applicable, and the effect which the practitioner desires the bispecific antibody to have upon a pathogenic antigenic molecule or autoantibody.

It is also understood that appropriate doses of bispecific antibodies depend upon the potency of the bispecific antibody with respect to the antigen to be cleared. Such appropriate doses may be determined using the assays described herein. When one or more of these bispecific antibodies is to be administered to an animal (e.g., a human) in order to clear an antigen, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the bispecific antibody employed, the age, body weight, general health, gender, and diet of the subject, the time of administration, the route of administration, the rate of excretion, any drug combination, and the concentration of antigen to be cleared.

### 5.6 Kits

The invention also provides kits containing the bispecific molecules of the invention. Kits containing the pharmaceutical compositions of the invention are also provided.

### 6. EXAMPLES

The following examples describe the production of bispecific molecules comprising an anti-CR1 mAb and an antibody that binds the protective antigen (PA) protein of Bacillus anthracis (Anthrax), a common component of the lethal and edema toxins of Anthrax (see, e.g., Little et al., 1991, Biochem Biophys Res Commun. 180:531-7; Little et al., 1988, Infect Immun. 56:1807-13). It was shown that binding of PA to cell receptors is required for toxicity (see, e.g., Little et al., 1988, Infect Immun. 56:1807-13). The 14B7 antibody binds PA (see, e.g., Little et al., 1991, Biochem Biophys Res Commun. 180:531-7; Little et al., 1988, Infect Immun. 56:1807-13). The bispecific molecules produced in the Examples can therefore be used for treatment of Anthrax infection by removing PA from the circulation.

In particular, Example 6.1 describes the production of bispecific molecules comprising an anti-CR1 mAb, 7G9, and an anti-PA antibody, 14B7IgG, using N-succinimidyl-S-acetyl-thioacetate (SATA) and N-hydroxysuccinimide-poly(ethylene glycol)-maleimide (NHS-PEG-MAL) as the cross-linking agents. Example 6.2 describes the production of bispecific molecules comprising 7G9 and an anti-PA single chain antibody, 14B7scAb, using N-succinimidyl-S-acetyl-thioacetate (SATA) and NHS-poly(ethylene glycol)-maleimide (PEG-MAL) as the cross-linking agents; Example 6.3 describes the production of bispecific molecules comprising 7G9 and 14B7Fab using N-succinimidyl-S-acetyl-thioacetate (SATA) and NHS-poly(ethylene glycol)-maleimide (PEG-MAL) as the cross-linking agents; and Examples 6.4-6.6 describe the production and characterization of bispecific molecules comprising humanized monoclonal antibody H9 and the monoclonal antibody 14B7 using C6 4-hydrazino-nictoinamide acetone hydrazone (Hz) (Solulink) and NHS-PEG-benzaldehyde as the crosslinking agents.

### Example 6.1

### Bispecific Molecules 14B7IgG-PEG-7G9IgG

Bispecific molecules comprising an anti-CR1 monoclonal antibody, 7G9, and an anti-PA antibody, 14B7IgG were produced, and are herein referred to as 14B7IgG-PEG-7G9IgG, for simplicity.

Anti-CR1 Monoclonal Antibody, 7G9

A hybridoma cell line secreting a high-affinity anti-CR1 monoclonal antibody was used to produce the 7G9 (murine IgG₂ₐ, kappa) anti-CR1 mAb. A master cell bank (MCB) was generated from this cell line and tested (Charles River Tektagen) for mouse antibody production, mycoplasma and sterility. The 7G9 antibody used in the production of the bispecific molecules was produced and purified from ascites fluid from mice.

Derivitization/Cross-Linking

The anti PA-antibody, herein referred to as the 14B7IgG antibody, was derivatized with SATA using the following protocol. The 14B7IgG antibody was dialyzed overnight in PBSE buffer at 4° C. After dialysis, the volume of 14B7IgG was 1.8 ml, and the protein concentration was 4.3 mg/ml as determined by A280 measurement. SATA (MW 231.2 g/mol) stock solution was prepared at 3.5 mg/ml in DMSO. 7.2 ul of the SATA stock solution (0.025 mg, 108 nmoles) was added to 18 nmoles (2.7 mg; volume=0.628 ml) of dialyzed 14B7IgG (at a 6×:1×molar ratio; 6×SATA: 1×14B7IgG) and reacted at room temp for 2 hours with gentle inversion every 15-30 minutes. Hydroxylamine HCl (“HA-HCl”; MW 69.49 g/mol) stock solution was prepared by adding 0.76 g hydroxylamine HCl and 1.0 ml 0.5 M EDTA to 25 ml MES at pH 7.5 (38.8 mg/ml). 72 ul of the HA-HCl solution was (2.79 mg, 36 umoles) added to the reaction mixture of the SATA-derivatized 14B7 IgG (at a molar ratio of 2000×:1×; 2000×HA-HCl to 1×14B7IgG-SATA derivatized) and reacted at room temp for 2 hours under argon gas. The mixture was subsequently desalted by chromatography over an Amersham Hi-Prep desalting column (26/10) in MES buffer (Volume of pool=3.8 ml, protein concentration as determined by A280 is 0.57 mg/ml, 67% to 80% recovery)

The anti-CR1 monoclonal antibody, the 7G9 antibody was derivatized with NHS-PEG-maleimide. NHS-PEG-maleimide derivitization of 7G9 antibody resulted in 68% recovery of 7G9IgG-PEG (NHS-PEG-maleimide is obtained from Shearwater Corporation and the catalog number for the Shearwater PEG is: 2D2Z0F021). NHS-PEG maleimide (MW 3400 g/mol) stock solution was prepared at 50 mg/ml in MES buffer (14.7 nmoles/ul). 7.34 ul of the NHS-PEG maleimide stock solution was added to the 7G9 antibody at a molar ratio of 8:1 (8×NHS-PEG maleimide to 1×7G9 antibody) and reacted at room temp 2 hours with gentle inversion every 15-30 minutes. The mixture was then desalted by chromatography over an Amersham Hi-Prep desalting column (26/10) in MES buffer. For NHS-PEG-maleimide derivatization various molar ratios were used in order to determine optimal molar ratios for the derivitization protocol.

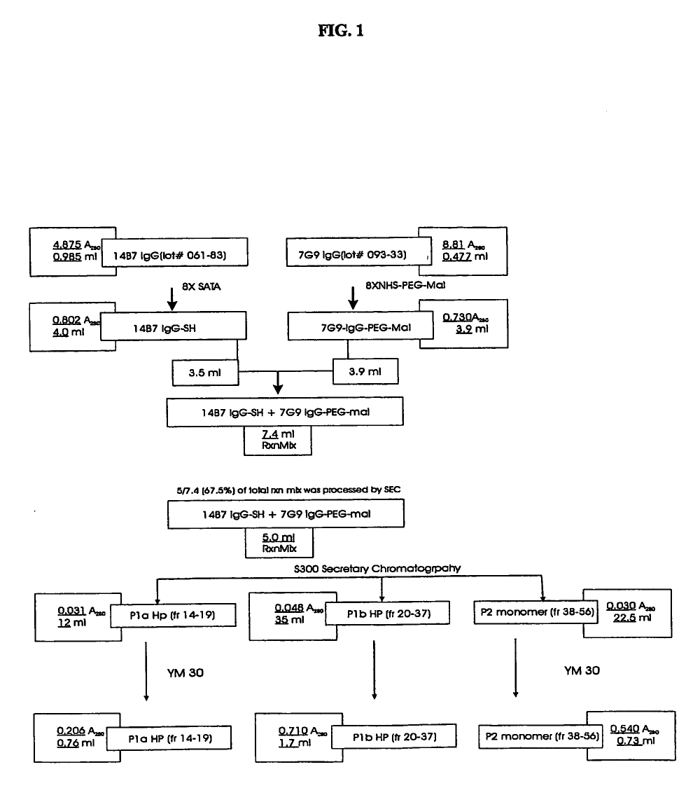

The deriviatized antibodies were combined at equal mass in the cross-linking reaction mixture. The total protein in the final reaction mixture was not determined by lowry or an A₂₈₀ measurement. The total protein was assumed to be sum of the input antibodies (3.8 mgs), and the final volume of the reaction mixture was 7.4 ml. A flow chart showing the cross-linidng and derivatization process involved in making the 14B7IgG-PEG-7G9IgG is shown in FIG. 1.

Sephacryl 300 Size Exclusion Chromatography (“SEC”) Fractionation

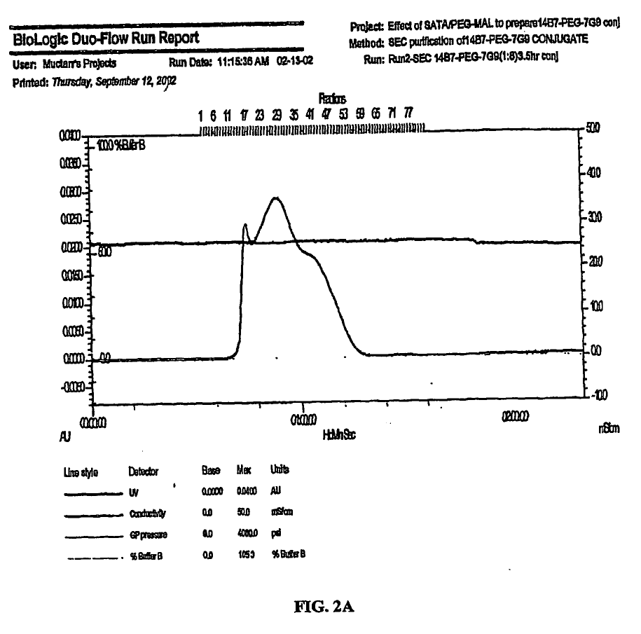

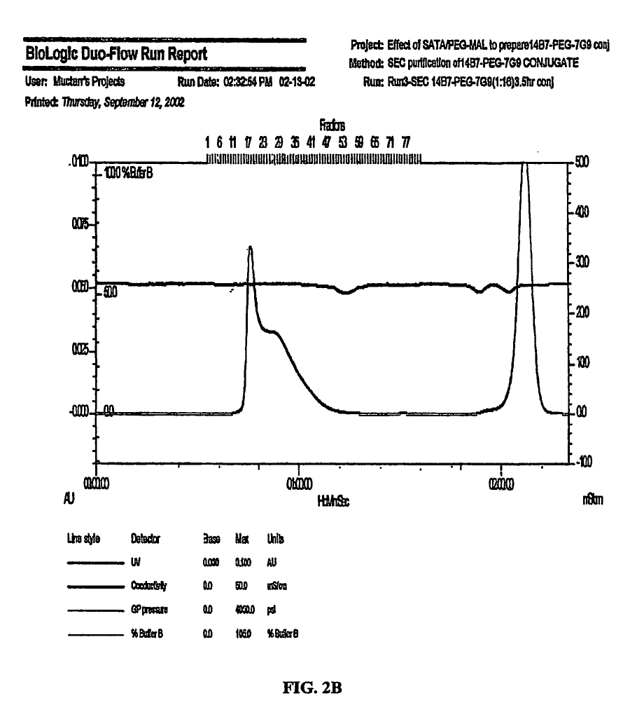

A 5 ml (2.6 mgs) portion (68%) of the final reaction mixture was processed further by fractionation on Sephacryl 300. The elution profile for the reaction mixture resolved into three peak areas that were collected as fractions as shown in FIG. 2. Column fractions were combined into pools according to the peaks of the elution profile. A discrete void volume peak, fractions 14 through 19 with a total volume of 12 ml, was labeled as the High Molecular Weight; “HMW” fraction. A second, broad, predominant peak, fractions 20 through 37, with a total volume of 35 ml, was labeled Low Molecular Weight “LMW” fraction. A third shoulder peak, fractions 38 through 56, with a total volume of 22.5 ml, was labeled Monomer fraction. The three pooled peak fractions were analyzed for protein concentration by measuring A₂₈₀. The total reaction mixture (7.4 ml) contained 3800 micrograms of input antibody. The 5-ml portion fractionated by S300 contained 2568 micrograms protein. The total protein recovered post SEC in all three fractions was 1818 micrograms (71%). Each S300 peak fraction was further processed by concentration then analyzed by A₂₈₀ (Table 1). The elution profile is shown in FIG. 2A. FIG. 2B represents the elution profile when the molar ratio for derivitization was 16:1 (16×NHS-PEG maleimide to 1×7G9 antibody).

TABLE 1
PROTEIN RECOVERY

| Item | Sample | 14B7IgG | 7G9IgG | Total | % | % |
|---|---|---|---|---|---|---|
| 1 | input Ab(ug) | 1916 | 1888 | 3804 | 100 | |
| 2 | derivitized Ab(ug) | 1284 | 1284 | 2568 | 68 | |
| 3 | input Rxn Mig(ug) | | | 2568 | 68 | 100 |
| 4 | S300(Fr14-19) | | | 248 | 7 | 10 |
| 5 | S300(Fr20-37) | | | 1120 | 29 | 44 |
| 6 | S300(Fr38-56) | | | 450 | 12 | 18 |
| 7 | Conc(Fr14-19) | | | 104 | 3 | 4 |
| 8 | Conc(Fr20-37) | | | 805 | 21 | 31 |
| 9 | Conc(Fr38-56) | | | 263 | 7 | 10 |

The final concentrated fractions were evaluated by Lowry, CAA and HPCA for specific activity measurements as shown in Table 2.

TABLE 2
CHARACTERIZATION OF SEC FRACTIONS

| | | Total | Total | Total | CAA/ | HFCA/ |
|---|---|---|---|---|---|---|
| Item | Sample | Protein | CAA | HPCA | Protein | Protein |
| 1 | Conc(Fr14-19) | 94 | 21 | 163 | 0.22 | 1.73 |
| 2 | Conc(Fr20-37) | 877 | 350 | 1698 | 0.40 | 1.94 |
| 3 | Conc(Fr38-56) | 288 | 111 | na | 0.39 | na |
| 4 | control 7G9 | 587 | 899 | | 1.53 | na |
| | IgG(ug) | | | | | |

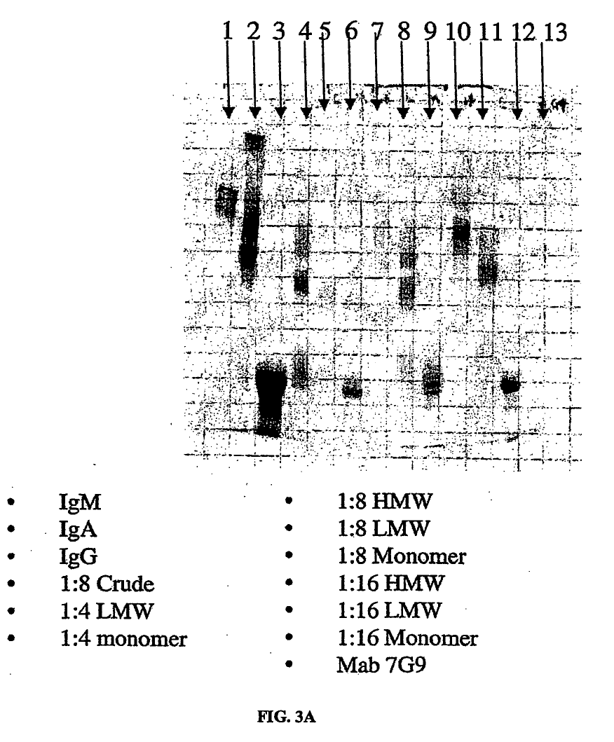

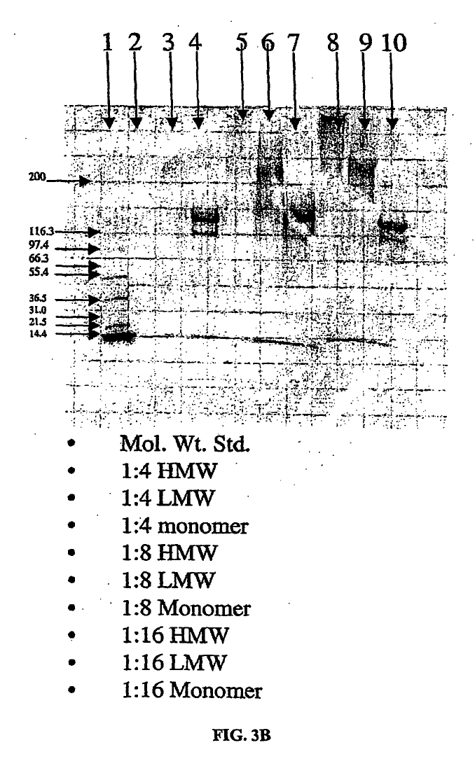

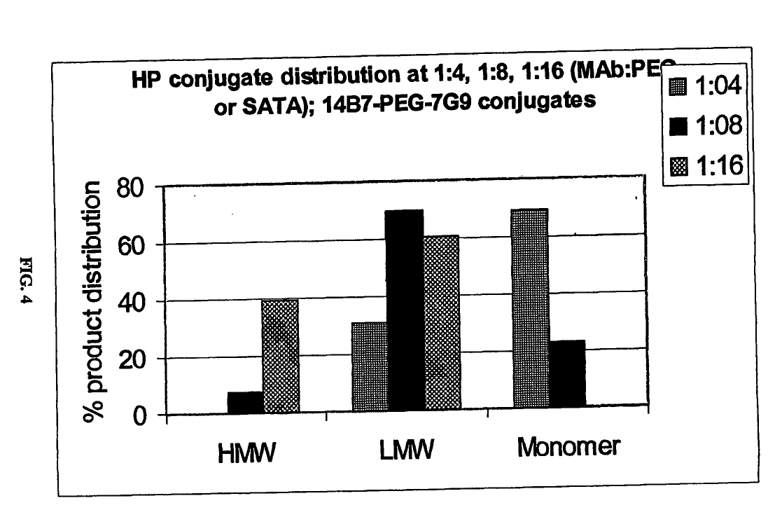

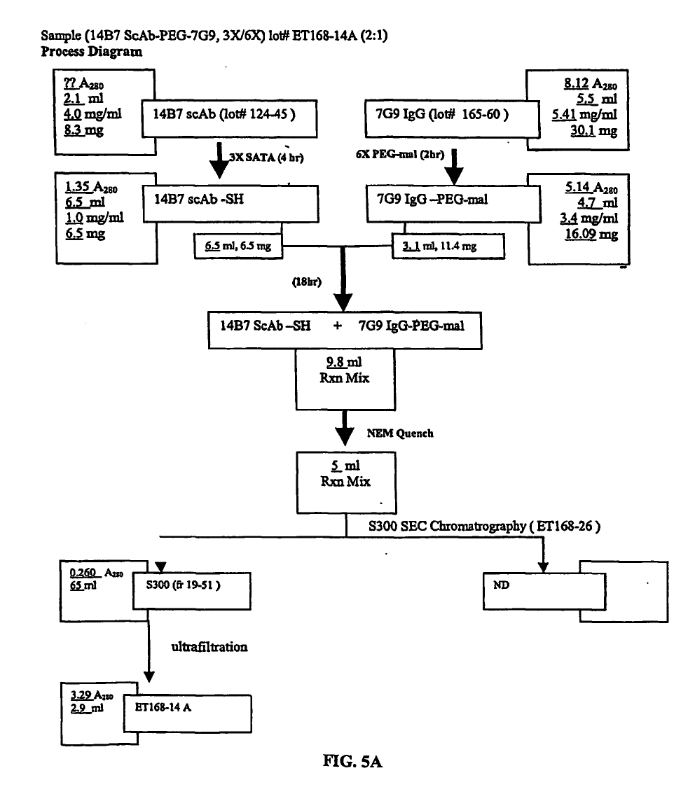

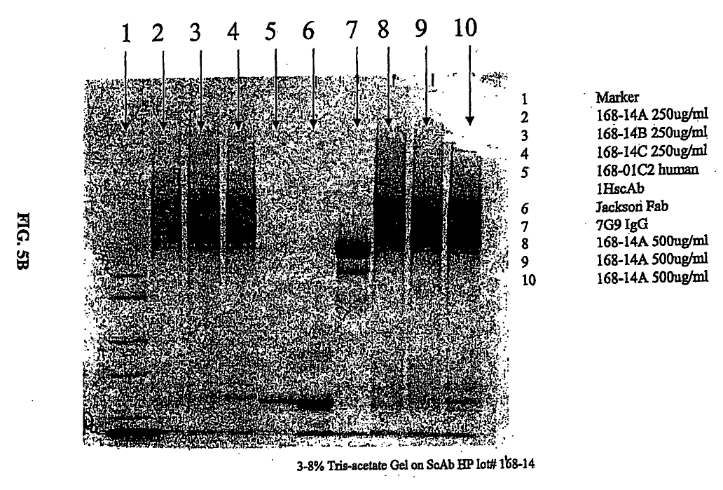

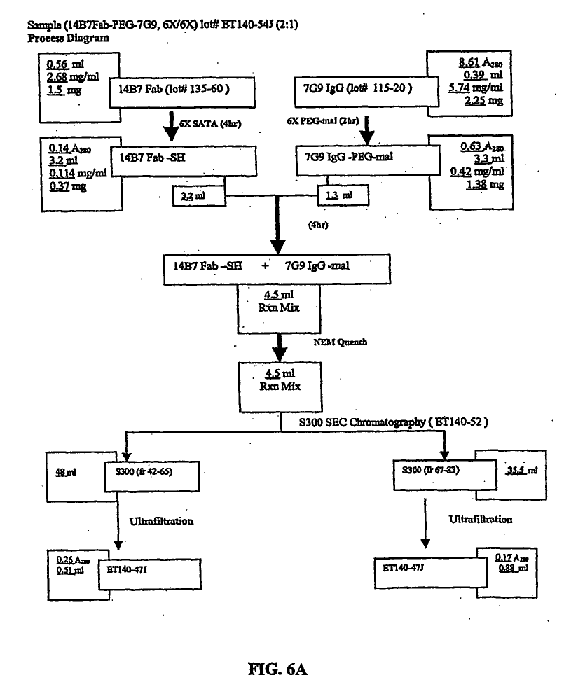

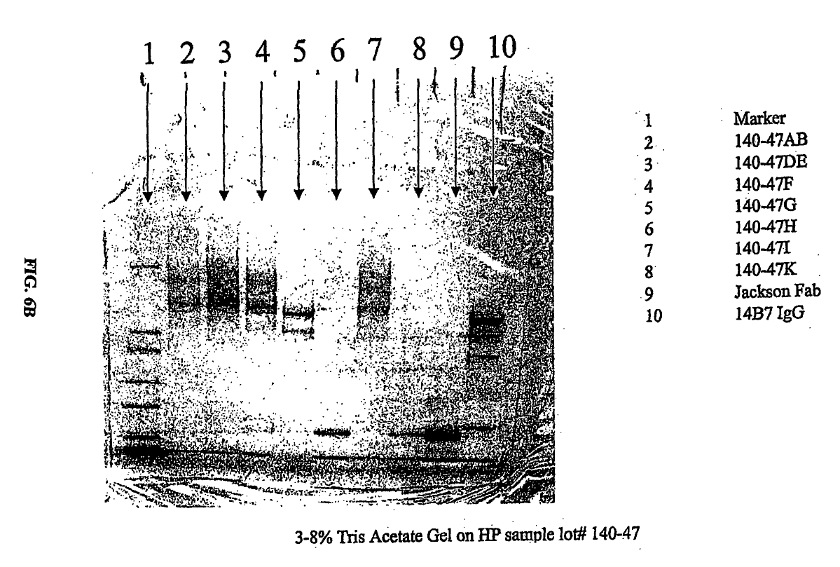

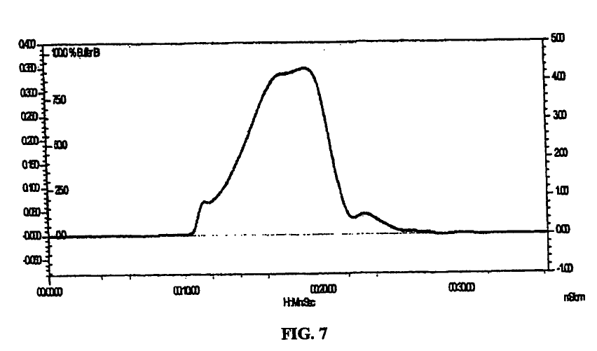

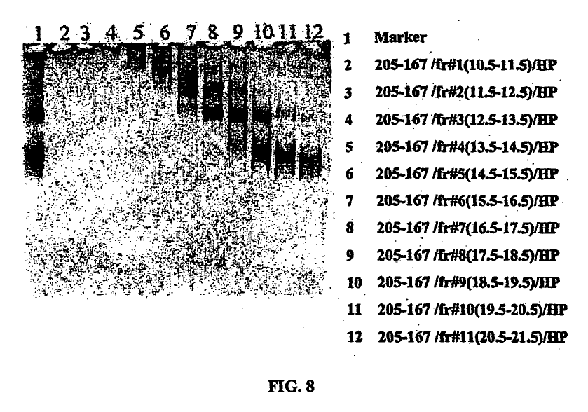

The % molecular weight distribution by SDS-PAGE of each species was estimated visually from the gel on each S300 fraction as presented in Table 3 and is shown also in FIG. 3. FIG. 3 shows the distribution of molecular weight species of 14B7IgG-PEG-7G9IgG as produced by different conjugations of NHS-PEG-maleimide to 7G9 based on their mobilities on SDS-PAGE.

TABLE 3
MOLECULAR WEIGHT DISTRIBUTION BY S300
FRACTIONATION

| | | | Protein | | | % MW Distribution | | | |
|---|---|---|---|---|---|---|---|---|---|
| | S300 | Fraction | Lowry | Total | % | (SDS-PAGE) | | | |
| Item | Fractions | Vol(ml) | (ug/ml) | ug | Load | mono | dimer | tri | tetra |
| 1 | S300 Load | 5 | | 2568 | 100 | 30 | 30 | 30 | 10 |
| 2 | S300(Fr14-19) | 0.76 | 124.0 | 94 | 4 | | | | 100 |
| 3 | S300(Fr20-37) | 1.7 | 516.0 | 877 | 34 | 5 | 50 | 45 | |
| 4 | S300(Fr38-56) | 0.73 | 395.0 | 288 | 11 | 95 | 5 | | |
| 5 | Total | | | 1260 | 49 | | | | |

TABLE 4
MOLECULAR WEIGHT DISTRIBUTION BY S300
FRACTIONATION

| | | | Protein | | | % MW Distribution | | | | |
|---|---|---|---|---|---|---|---|---|---|---|
| | S300 | Fraction | Lowry | Total | % | (SDS-PAGE) | | | | |
| Item | Fractions | Vol(ml) | (ug/ml) | ug | Load | mono | dimer | tri | tetra | |
| 1 | S300 Load | 5 | | 2568 | 100 | 770 | 770 | 770 | 257 | |
| 2 | S300(Fr14-19) | 0.76 | 124.0 | 94 | 4 | | | | 94 | |
| 3 | S300(Fr20-37) | 1.7 | 516.0 | 877 | 34 | 44 | 439 | 395 | | |
| 4 | S300(Fr38-56) | 0.73 | 395.0 | 288 | 11 | 274 | 14 | | | |
| 5 | | | | | Total | 318 | 453 | 395 | 94 | 1260 |
| 6 | | | | | % Load | 12 | 18 | 15 | 4 | 49 |

The PEG conjugation procedure produced a population of 14B7IgG-PEG-7G9IgG molecules that contained multiple molecular weight species. Analysis of the reaction mixture by SEC and SDS-PAGE shows the following molecular distribution: 37% Product (di, tri, tetra), and 12% Monomer.

Tables 5 & 6 further summarize the characterization of each species produced in the production of 14B7IgG-PEG-7G9IgG as characterized by Lowry, A280 measurements, and CAA assays. It should be noted that the results presented show the results of various NHS-PEG-maleimide conjugations for the production of 14B7IgG-PEG-7G9IgG. Specifically, 14B7IgG-PEG-7G9IgG was produced using a 1:4, 1:8, and 1:16 molar ratio, each of which were characterized (1:4 meaning 1×NHS-PEG-maleimide:4×7G9IgG; 1:8 meaning 1×NHS-PEG-maleimide:8×7G9IgG; 1:16 meaning 1×NHS-PEG-maleimide: 16×7G9IgG). FIG. 4 further summarizes the distribution of molecular weight species of 14B7IgG-PEG-7G9IgG as produced by different conjugations of NHS-PEG-maleimide to 7G9IgG.

TABLE 5
CHARACTERIZATION OF 14B7IGG-PEG-7G9IGG

| ID | Lowry (ug/ml) | CAA (ug/ml) | CAA/Lowry |
|---|---|---|---|
| 1:4 HMW | ND | ND | N/A |
| (Fraction 15-18) | | | |
| 1:4 LMW | 83.5 | 21.7 | 0.26 |
| (Fraction 19-36) | | | |
| 1:4 Monomer | 245.0 | 97.3 | 0.40 |
| (Fraction 37-58) | | | |
| 1:8 HMW | 124.1 | 23.5 | 0.19 |
| (Fraction 14-19) | | | |
| 1:8 LMW | 515.5 | 156.7 | 0.30 |
| (Fraction 20-37) | | | |
| 1:8 Monomer | 395.3 | 116.6 | 0.29 |
| (Fraction 38-56) | | | |
| 1:16 HMW | 733.5 | 133.5 | 0.18 |
| (Fraction 16-23) | | | |
| 1:16 LMW | 488.8 | 68.3 | 0.14 |
| (Fraction 24-40) | | | |
| ET093-33 7G9 | *5873.3 | 8986.3 | 1.53 |
| (monomer) | | | |

TABLE 6
CHARACTERIZATION OF 14B7IGG-PEG-7G9IGG

| | A280 | Lowry | Total Protein | Distribution by | Total Protein | Distribution by |
|---|---|---|---|---|---|---|
| ID | (ug/ml) | (ug/ml) | A280 (ug) | A280 (%) | Lowry (ug) | Lowry (%) |
| 1:4 HMW | 7.6 | ND | 5.0 | 1.5 | N/A | N/A |
| (Fraction 15-18) | | | | | | |
| 1:4 LMW | 84.7 | 83.5 | 110.0 | 32.0 | 108.6 | 30.7 |
| (Fraction 19-36) | | | | | | |
| 1:4 Monomer | 230.2 | 245.0 | 230.0 | 66.5 | 245.0 | 69.3 |
| (Fraction 37-58) | | | | | | |
| 1:8 HMW | 137.5 | 124.1 | 105.0 | 9.3 | 94.3 | 7.0 |
| (Fraction 14-19) | | | | | | |
| 1:8 LMW | 473.5 | 515.5 | 805.0 | 71.0 | 876.4 | 70.0 |
| (Fraction 20-37) | | | | | | |
| 1:8 Monomer | 360.7 | 395.3 | 224.0 | 19.7 | 288.6 | 23.0 |
| (Fraction 38-56) | | | | | | |
| 1:16 HMW | 649.9 | 733.5 | 481.0 | 40.0 | 542.8 | 39.5 |
| (Fraction 16-23) | | | | | | |
| 1:16 LMW | 428.4 | 488.8 | 728.0 | 60.0 | 831.0 | 60.5 |
| (Fraction 24-40) | | | | | | |

Note:
ND = Not detected
* = By A280

CR1 Antibody Assay (“CAA”) and Heteropolymer Conjugate Assay (“HPCA”)

The functionality of the 14B7IgG-PEG-7G9IgG bispecific molecules were determined using the CAA assay or the HPCA assay as described.

CAA Assay

This assay can be used generally to measure any anti-CR1 antibody or any molecule comprising an anti-CR1 antibody. ELISA/CR1 plates were prepared by incubating ELISA plates, high binding flat bottom ELISA plates (Costar EIA/RIA strip plate 2592) with a suitable amount of a bicarbonate solution of CR1 receptors. The concentration of the bicarbonate solution of CR1 receptors was 0.2 ug/ml prepared from a 5 mg/ml CR1 receptors stock (Avant Technology Inc.) in a carbonate-bicarbonate buffer (pH 9.6, Sigma C-3041). 100 ul CR1-bicarbonate solution was dispensed into each well of the ELISA plates and the plates were incubated at 4° C overnight. The plates were then washed using, a wash buffer containing PBS, 0.1% Tween-20, and 0.05% 2-Chloroacetamide. A SuperBlock Blocking Buffer in PBS (Pierce) was added to the plates for about 30-60 min at room temperature after the wash. The plates were dried and stored at 4° C. A human anti-CR1 IgG having a concentration of 300 or 600 mg/ml was used as the control, or “calibrator”. The composition of 14B7IgG-PEG-7G9 was titrated carried using PBS, 0.25% BSA, 0.1% Tween-20 as the diluent buffer, PBS, 0.1% Tween-20, 0.05% 2-Chloroacetamide as the wash buffer, TMB-Liquid Substrate System for ELISA (3,3′,5.5′-Tetramethyl-Benzidine) and 2N H₂SO₄ as the stop solution.

HPCA Assay

The HPCA assay was used to analyze the functionality of the cross-linked anti-PA bispecific molecule 14B7IgG-PEG-7G9 in terms of the binding specificity of 14B7IgG-PEG-7G9 to CR-1 and PA.

Materials and Methods:

Plates (Corning Costar Assay plate, v-bottom non-treated polystyrene) were coated with CR-1 at a concentration of 0.2 ug/ml. An anti-PA heteropolymers was used as an internal standard, 14B7x7G9 at a concentration of 464.0 μg/ml.

HC=1.0 μg/ml, MC=0.5 μg/ml, LC=0.25 μg/ml Biotin conjugated PA was used at a concentration of 0.81 mg/ml. The ELISA Diluent Buffer contained 1×PBX buffer, 0.25% BSA, 0.1% Tween 20, 0.05% 2-Chloroacetamide. The ELISA Wash Buffer contained 1×PBS, 0.1% Tween-20, 0.05% 2-Chloroacetamide. TMB was obtained from Sigma (cat #T-0440, LOT #21K1392). The stop solution contained 2N H₂SO₄. Horse radish Peroxidase-conjugated Streptavidin; SA-HRP was provided at 0.5 mg/ml.

Initially the cross linked heteropolymer was bound to the CR-1 plate using the following procedure. The heteropolymer was diluted to 5 μg/ml in the ELISA diluent buffer. In a dilution plate, samples were loaded at 5 μg/ml in rows A through H and serially diluted 1:3 fold. All samples were run in duplicates including calibrators.

100 μl of diluted samples were transferred from the dilution plate into corresponding wells on the CR-1 coated plate. 100 μl of HC, MC, and LC were added in duplicates to rows A11 and A12, B11 and B12, C11 and C12, respectively. 100 μl of diluent was added for blanks to five wells in duplicates. The plate was then sealed with the adhesive plate sealer and incubated at 37° C. for 1 hour. The solution was discarded and the plate was washed on auto plate washer with 5-cycle program.

Next biotinylated PA (“b-PA”) was bound to the heteropolymer using the following procedure. b-PA was diluted to 2.5 ng/ml in ELISA diluent buffer. 100 μl of diluted b-PA was transferred into all wells (including blank wells). The plate was then sealed with the adhesive plate sealer and incubated at 37° C. for 1 hour. The solution was discarded and the plate was washed on auto plate washer with 5-cycle program.

Finally streptavidin conjugated horseradish peroxidase (“SA-HRP”) was bound to b-PA using the following method. SA-HRP was diluted 1:10,000 in ELISA diluent buffer.

100 μl of diluted SA-HRP was transferred into all wells (including blank wells). The plate was then sealed with the adhesive plate sealer and incubated at 37° C. for 1 hour. The solution was discarded and the plate was washed on auto plate washer with 5-cycle program.

In order to develop signal, 100 μl of pre-warmed TMB was added to all wells. The plate was incubates at room temperature for 15 min (protected from light). 100 μl of stop solution (2N H₂SO₄) was addend, and the plate was additionally incubates at room temperature for another 10 min. The plate was read at 450 nm using a plate reader.

The majority of the CAA and HPCA activity was in the predominant S300 pool from peak 2. This data is summarized in Table 5.

TABLE 7
14B7IGG-PEG-7G9IGG CHARACTERIZATION

| | Fraction | Lowry Conc. | | | HPCA1 | HPCA/ |
|---|---|---|---|---|---|---|
| ID | # | (ug/ml) | CAA (ug/ml) | CAA/Lowry | (μg/ml) | Lowry |
| 1:4 HMW | 15-18 | ND | ND | N/a | NA | NA |
| 1:4 LMW | 19-36 | 83.5 | 27.6 | 0.33 | 168.49 | 1.99 |
| 1:4 Monomer | 37-58 | 245 | 105.9 | 0.43 | NA | NA |
| 1:8 HMW | 14-19 | 124.1 | 26.8 | 0.22 | 215.43 | 1.57 |
| 1:8 LMW | 20-37 | 515.5 | 205.8 | 0.4 | 999.4 | 2.11 |
| 1:8 Monomer | 38-56 | 395.3 | 152.4 | 0.39 | NA | NA |
| 1:16 HMW | 16-23 | 733.5 | 169 | 0.23 | 738.32 | 1.14 |
| 1:16 LMW | 24-40 | 488.8 | 77.4-208.2 | 0.16-0.43 | 350.8 | 0.82 |
| ET093-33 7G9 | — | 5783.3 | 9169.3 | 1.56 | ND | ND |
| | | nb112pg03 | nb119pg11 | | nb89pg79 | |

### Example 6.2

### Bispecific Molecules 7G9-PEG-14B7scAb

In this example, the anthrax PA binding antibody fragment was a single chain antibody fragment consisting of a single chain Fv of murine monoclonal antibody 14B7 fused with a human constant k domain The scAb fragment was prepared according to the procedure described in Maynard et al., Nature Biotechnology 20:597-601. A flow chart showing the production process is depicted in FIG. 5A.

The 14B7scAb antigen-binding antibody fragment was derivatized with SATA as described in Example 6.1. 14B7scAb was derivatized using a molar ratio of 1:3 (14B7scAb:SATA).

The 7G9 antibody was derivatized with NHS-PEG-MAL (Shearwater Polymers, Cat. #2D2Z0F021) as follows. A 50 mg/ml MES solution of NHS-PEG-MAL (14.7 nmol/ul) was prepared. 7.34 ul of the NHS-PEG-MAL solution was added to 1.5 ml 7G9 (36 nmol) (molar ratio of about 3:1 PEG:antibody). The reactants was incubated at room temperature for about 2 hours with gentle inversion every 15-30 min. The reaction mixture is then desalted by chromatography using an Amersham Hi-Prep desalting column in MES buffer. The reaction mixture was then desalted by chromatography using an Amersham Hi-Prep desalting column (26/10) in MES buffer. 3.3 ml of pooled sample was recovered. The recovered sample was 1.5 mg, and had a protein concentration of 0.45 mg/ml (A280), representing a 3.3% recovery. The PEG-MAL modified antibody 7G9-PEG-MAL was eluted in the void volume with PBSE buffer.

A reaction mixture of 14B7scAb-SH and 7G9-PEG-MAL with a molar ratio of 2:1 (14B7Fab-SH:7G9-PEG-MAL) was prepared. The reaction mixtures were incubated for 18 hours. The mixture was quenched in NEM and fractioned using S300 SEC chromatography the next day.

Sample ET168-14A was a pool of fractions from an S300 column run. The S300 column run (ET168-26), loaded with 5-ml concentrated reaction mixture, generated 120, 2-ml fractions. A 65-ml pool from fractions 19 through 51 was labeled as ET168-14A . The pooling process was recorded on ET168-26. Sample ET168-14A was further processed by ultrafiltration to concentrate the product mixture to a final volume of 2.9 ml. SDS-PAGE analysis shows sample ET168-14A contains 10% free scAb, 45% monomer (PEG-7G9) and 45% higher MW bispecific molecules. FIG. 5B shows a photograph of a Tris-Glycine SDS PAGE containing the sample ET168-14A.

SDS-PAGE, functional CR1binding (CAA), functional PA binding (PAA), bivalency binding (HPCA) and protein content (Lowry) data for samples ET168-14A are summarized in Table 8.

Lowry data show that 9.3 milligrams of protein was recovered in the final bispecific molecule mixture, 168-14A. This represents a 32% of the total starting input antibody (28 milligrams). SDS-PAGE analysis shows sample 168-14A contained multiple conjugated species and approximately 45% non-cross linked antibodies. SDS-gel shows conjugate size of approximately 200 kD. At 200 kD expected molar ratio of 1:1 (ScAb:7G9).

Sample ET168-14A had CR1 binding activity as indicated by the CAA assay. Specific activity was calculated at 0.58.

The sample ET168-14A demonstrated anthrax PA binding activity as indicated by the PAA assay. Specific activity was calculated 0.18 and the comparison to reference 14B7 antibody indicated approximately (0.18/0.71) 25% of the activity of an unmodified antibody. Specific activity of unmodified scab is not recorded.

The sample, ET168-14A, demonstrated bivalent binding activity indicating successful crosslinking of the two functional components, as indicated by the HPCA assay.

TABLE 8
CHARACTERIZATION OF ET168-14A

| | ET168-14A |
|---|---|
| HPCA C₅₀ value (mg/ml) | 0.166 |
| Max OD | 2.895 |

### Example 6.3

### Bispecific Molecules 7G9-PEG14B7Fab

In this example, the production of bispecific molecule 7G9-PEG-14B7Fab is described. A flow chart showing the production process is depicted in FIG. 6A.

The 14B7Fab antigen-binding antibody fragment was derivatized using SATA as described in Example 6.1. The 7G9 antibody was derivatized with NHS-PEG-MAL as described in Example 6.2.

A reaction mixture of 14B7scAb-SH and 7G9-PEG-MAL with a molar ratio of 2:1 (14B7Fab-SH:7G9-PEG-MAL) was prepared. The reaction mixtures were incubated for 4 hours. The mixture was quenched in NEM and fractioned using S300 SEC chromatography after two days.

Sample ET140-47I was pooled fractions from the S300 column run of the reaction mixture. The S300 column run, loaded with 4.5-ml reaction mixture, generated 140, 2-ml fractions. A 68-ml pool from fractions 24 through 57 was labeled ET140-54D. A 65-ml pool from fractions 42-64 was labeled ET140-47I. Sample ET140-47I was further processed by ultrafiltration to concentrate the preparations to a final volume of 0.5 ml. SDS-PAGE analysis shows that sample D contains free antibodies and higher MW bispecific molecules. FIG. 6B shows a photograph of a Tris-Glycine SDS PAGE containing the sample ET140-47I.

SDS-PAGE, functional CR1 binding (CAA), functional PA binding (PAA), bivalency binding (HPCA) and protein content (Lowry) data for samples ET140-47I are summarized in Table 9.

Lowry data show that 0.070 milligrams of protein was recovered in the bispecific molecule fraction, 140-47I. This represents a 3% of the total starting input antibody (2.4 milligrams). SDS-PAGE analysis shows that sample D contained multiple conjugated species and approximately 50% unreacted antibodies.

Sample ET140-47I had CR1 binding activity as indicated by the CAA assay. Specific activity was calculated at 0.33 and the comparison to reference 7G9 antibody indicated approximately 39% (0.33/0.85) of the unmodified antibody activity.

Sample ET140-47I demonstrated anthrax PA binding activity as indicated by the PAA assay. Specific activity was calculated 0.07. Specific activity of unmodified 14B7 was not recorded.

Sample ET140-47I demonstrated bivalent binding activity indicating successful crosslinking of the two functional components, as indicated by the HPCA assay.

TABLE 9
CHARACTERIZATION OF ET140-47I

| | ET140-47I |
|---|---|
| HPCA C₅₀ value (mg/ml) | 0.217 |
| Max OD | 1.419 |

### Example 6.4

### Synthesis of N-Hydroxysuccinimidyl-Polyethylene Glycol-Benzaldehyde. (PBA) (1)

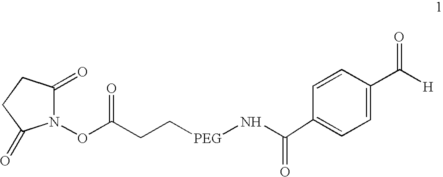

In a 25-mL round bottomed flask, 500 mg of carboxyl-polyethylene glycol-amine (0.147 mmole) (Shearwater) was diluted with 25 ml of 10 mM phosphate buffer, pH 7.5. To the resulting solution was added 49.42 mg of N-hydroxysuccinimidyl-formylbenzoate (Solulink) which had been dissolved in dimethyl sulfoxide. The resulting reaction was stirred at room temperature under argon in the dark. After 4 hours, the aqueous phase was extracted with dichloromethane (DCM). The DCM phase was dried over MgSO₄ and concentrated under reduced pressure to provide a residual liquid which was extracted with ether (3×50 mL). Carboxy-PEG-benzaldehyde (CPB) was precipitated by adding cold isopropyl alcohol (IPA) to the combined ethereals. The precipitate was then washed with cold IPA then dissolved in 8 ml of DCM. To the resulting solution was added 0.8 ml of 10% of sodium phosphate buffer at pH 5.0, followed by 150 mg of(1-ethyl-3-(3-dimethylamino propyl)carbodiimide (EDC), and 102 mg of N-hydroxysuccinimide (NHS). The resulting reaction was stirred under argon for 2 hours, the DCM phase was collected, dried over MgSO4 and concentrated in vacuo to provide an oily residue which was washed using IPA and dried in vacuo to provide compound 1 (yield=238 mg). The molecular weight of PEG is 3400 Da

The residual carboxyl group in the intermediate product was completely converted to the final product by another reaction with EDC and NHS. For instance, 50 mg of the intermediate product was dissolved in 2.5 ml of ethyl acetate. 16.11 mg of NHS and 28.475 mg of EDC were added. The reaction mixture was stirred for 1.5 hours under Argon. The reaction mixture was concentrated down to a colorless gumlike material. Two ml of ether was added to allow a precipitate to form. Ether was decanted and the residue was washed with ether for two more times. A solid material (23 mg) was collected as the final product. The compound was analyzed on a thin layer chromatography plate and was obeserved as a distinct spot. The ultraviolet spectrum of the final product was identical to NHS-benzaldehyde. This compound (N-hydroxy-succinimidyl-polyethylene glycol-Benzaldehyde) hereafter will be referred to as PBA and has Formula I.
N-hydroxy-succinimidyl-polyethylene glycol-Benzaldehyde) (PBA) (1)

### Example 6.5

### Bispecific Molecule: H9-PEG-Benzhydrazone-Nicotinate-Capryl-14B7 (HZ-HP)

The humanized monoclonal antibody H9 was derivatized with the bifunctional polymeric NHS-PEG-benzaldehyde (PBA). The monoclonal antibody 14B7 was derivatized with the bifunctional compound succinimidyl C6 4-hydrazino-nicotinamde acetone hydrazone (Hz) (Solulink). 500 nmoles of Hz was used to modify 31.25 nmole of 14B7 in a sample-buffer containing 0.1 5M NaCl, 50 mM potassium phosphate, pH 7.4. The reaction was stirred for 1 hour at room temperature. Small molecules were removed from the reaction mix in a 10 ml PD10-column (Amersham) which had been equilibrated with the conjugation buffer (0.1M citrate, pH 5). In a separate reaction, 500 nmoles of PBA was used to modify 31.25 nmole of H9 in the sample buffer as specified above. After 1 hour of stirring the reaction mixture at 25° C., small molecules were removed in a PD10 column.

The conjugation reaction was initiated by mixing the two monoclonal antibodies at a total protein concentration of 1-3 mg/ml, and the reaction was carried out for 16 hours at room temperature. The molar ratio of the two derivatized monoclonal antibodies during conjugation was 1:1. The crosslinked bispecific sample was then purified on a Suprose6 column (Amersham) which had been equilibrated with PBSG (10 mM phosphate, 0.15M NaCl, 5% glycerol, pH 7.4). The reaction product was separated into fractions of various molecular sizes depending on their elution. The total heteropolymeric protein generated was 46.7% of the starting material. The size exclusion profile is shown in the FIG. 7. The corresponding protein profile as analyzed by SDS-PAGE is shown in FIG. 8.

Depending on the elution time the apparent oligomeric state of the species was estimated. The fractions that eluted before 13.5 minutes correspond to highly crosslinked species. The fractions that eluted at 13.5 to 14.5 minutes correspond to teramers and pentamers. The fractions that eluted at 14.5 to 15.5 minutes correspond to trimers and tetramers. The fractions that eluted at 15.5 to 16.5 minutes correspond to trimers. The fractions that eluted at 16.5 to 17.5 minutes correspond to dimers and trimers. The fractions that eluted at 17.5 to 18.5 minutes correspond to dimers. The molecular weight distribution of the individual molecular species was 41.2% dimer, 32.5% trimer, 13% tetramer and 3.5% pentamer.

### Example 6.6

### Acitivity Assay of the Bispecific Molecule 14B7-HZ-PEG-H9

A bispecific molecule was produced using the same method as described above in Example 6.5, except that 6.25 nmoles of H9 was modified with 31.25 nmoles of NHS-PEG-Benzaldehyde (PBA). 6.25 nmoles of 14B7 was modified with 62.5 nmoles of Hz. The resulting heteropolymer mixture was resolved on a size exclusion Suprose6 column and 5 fractions corresponding to various forms of crosslinked molecules were collected and analysed by the above-mentioned activity assays, ELISA assays, such as CAA, PAA and HPCA in order to verify their binding activity. The result of the activity assays are summarized in the table below.

The activity of the bispecific molecule in each of the assays is dependent on the oligomeric state of the bispecific molecule, ie., the higher oligomeric state has reduced binding activity for the particular antigen assayed. This result may be a reflection of the binding property or stereo-availability of the heteropolymer to the antigen. The HPCA result clearly indicated that the bispecific molecule indeed has specificity for both CR1 and PA antigen since it demonstrated bivalent binding activity.

TABLE 10
Evaluation of PEG-Hydrazino-HP with various ELISA assays.

| Sample | PAA value | CAA value | HPCA |
|---|---|---|---|
| Dimer | 0.43 | 0.17 | 36 |
| Dimer-Trimer | 0.32 | 0.32 | 30 |
| Trimer | 0.26 | 0.22 | 9.4 |
| Trimer-Tetramer | 0.16 | 0.13 | 3.3 |
| Tetramer-pentamer | 0.05 | 0.07 | 0.97 |

### Example 6.7

### Synthesis of N-Hydroxy-Succinimidyl-PEG-Hydrazinonicotinate (2)

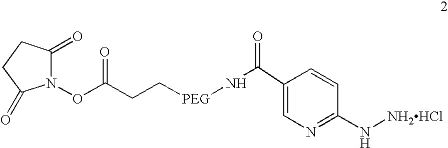

In a 25-mL round bottomed flask, 50 mg of carboxyl-polyethylene glycol-amine (0.0147 mmole) (Shearwater) is diluted with 2.5 ml of 10 mM phosphate buffer, pH 7.5. To the resulting solution is added 15.5 mg of N-hydroxysuccinimidyl-6-BOC-hydrazinonicotinate (Solulink) in tetrahydrofuran (THF). The resulting reaction is stirred at room temperature under argon in the dark. After 4 hours, the aqueous phase is extracted with dichloromethane (DCM). The DCM phase is dried over MgSO₄ and concentrated in vacuo to provide a liquid residue which is then precipitated using ether. The precipitated PEG-polymer is collected by filtration, and washed with 10 ml of cold (−20° C.) isopropyl alcohol (IPA) to provide carboxy-PEG-6-BOC-hydrazinonicotinate (CPN-Boc) which is then diluted with 1 ml of THF. To the resulting solution is added 15 mg of N,N′-dicyclohexylcarbodiimide (DCC), and 10.2 mg of N-hydroxysuccinimide (NHS) and the reaction mixture is stirred under argon for 3 hours. Dry silica gel (1 mg) is added to the solution and allowed to settle. The supernatant is separated from the solid precipitate and the solvent is then removed under vacuum to provide a residue which is resuspended in 0.5 ml of ethyl acetate, extracted with 0.5 ml of 3M HCl for 10 minutes (3 times). The organic phase is dried over MgSO₄, concentrated in vacuo and extracted with ether. Cold IPA is then added to the ethereal solution to provide compound 2 as a precipitate.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A bispecific molecule comprising:
(a) a first recognition binding moiety that binds a C3b-like receptor; and
(b) one or more second recognition binding moieties that binds a molecule; said molecule being other than a C3b-like receptor; wherein said first recognition binding moiety is cross-linked via a poly-(ethylene)glycol (PEG) linker to the second recognition binding moieties.

2. The bispecific molecule of claim 1, wherein said molecule is desired to be reduced in amount in the circulation of a mammal.

3. The bispecific molecule of claim 1, wherein the molecule is a pathogenic antigenic molecule.

4. The bispecific molecule of claim 3, wherein said pathogenic antigenic molecule is an autoimmune antigen.

5. The bispecific molecule of claim 1, wherein the molecule is an antigen of an infectious disease agent.

6. The bispecific molecule of claim 1, wherein said second recognition binding moiety is an antibody or an antigen binding antibody fragment thereof that binds an antigenic molecule.

7. The bispecific molecule of claim 6, wherein said antigen binding antibody fragment, is selected from a group consisting of Fab, Fab′, (Fab)′2, Fv or an sFv fragment.

8. The bispecific molecule of claim 1, wherein said second recognition binding moiety is a polypeptide, a peptide, an epitope, an antigenic determinant, a nucleic acid molecule, or a small molecule.

9. The bispecific molecule of claim 1, wherein said second recognition binding moiety binds the protective antigen (PA) protein of Bacillus anthracis (Anthrax).

10. The bispecific molecule of claim 1, wherein said poly-(ethylene) glycol (PEG) linker is a bifunctional poly-(ethylene)glycol (PEG) molecule, having the formula X-PEG-Y, wherein X and Y are functional groups.

11. The bispecific molecule of claim 1 or 10, wherein the poly-(ethylene)glycol (PEG) linker comprises a linear PEG molecule.

12. The bispecific molecule of claim 1 or 10, wherein the poly-(ethylene)glycol (PEG) linker comprises a non-linear PEG molecule.

13. The bispecific molecule of claim 12, wherein the non-linear poly-(ethylene)glycol (PEG) linker comprises a branched poly-(ethylene)glycol (PEG), linear forked poly-(ethylene)glycol (PEG), or branched forked poly-(ethylene)glycol (PEG) molecule.

14. The bispecific molecule of claim 10, wherein the X and Y functional groups are identical.

15. The bispecific molecule of claim 10, wherein the X and Y functional groups are different.

16. The bispecific molecule of claim 1, wherein said first and second recognition binding moieties comprise proteins, and wherein the bifunctional PEG linker derivatizes one or more amino acids within the first recognition binding moiety or the second recognition binding moieties.

17. The bispecific molecule of claim 16, wherein said amino acids are on the surface of the first or second recognition binding moieties.

18. The bispecific molecule of claim 17, wherein said amino acids are lysines, cyteines, histidines, serines, threonines, glutamic acids or arginines.

19. The bispecific molecule of claim 1, wherein said first and second recognition binding moieties comprise proteins, and wherein the bifunctional PEG linker derivatizes the N-terminal amino group of the first recognition binding moiety or the second recognition binding moieties.

20. The bispecific molecule of claim 1, wherein said first and second recognition binding moieties comprise proteins, and wherein the bifunctional PEG linker derivatizes the C-terminal carboxylic acid of the first recognition binding moiety or the second recognition binding moieties.

21. The bispecific molecule of claim 1, wherein said first recognition binding moiety that binds a C3b-like receptor is a monoclonal antibody that binds CR1.

22. The bispecific molecule of claim 21, wherein said second recognition binding moiety is cross-linked to the heavy or light chain of the first recognition binding moiety, with the proviso that said cross-linking is not via the carboxy terminus.

23. The bispecific molecule of claim 21, wherein said monoclonal antibody is a murine monoclonal antibody.

24. The bispecific molecule of claim 21, wherein said monoclonal antibody is a humanized monoclonal antibody.

25. The bispecific molecule of claim 1 or 10, wherein the molecular weight of the poly-(ethylene)glycol (PEG) molecule is 5 to 500 Daltons.

26. The bispecific molecule of claim 1 or 10, wherein the molecular weight of the poly-(ethylene)glycol (PEG) molecule is 200 to 20,000 Daltons.

27. The bispecific molecule of claim 1 or 10, wherein the molecular weight of the poly-(ethylene)glycol (PEG) molecule is 500 to 1000 Daltons.

28. The bispecific molecule of claim 1 or 10, wherein the molecular weight of the poly-(ethylene)glycol (PEG) molecule is 1000 to 8000 Daltons.

29. A method of producing a population of bispecific molecules, said method comprising contacting an antibody that binds a C3b-like receptor with one or more recognition binding moieties, wherein said antibody is conjugated with a bifunctional poly-(ethylene)glycol (PEG) linker, and wherein said one or more recognition binding moieties are derivatized to react with the bifunctional poly-(ethylene)glycol (PEG) linker, and wherein said one or more recognition binding moieties bind a molecule; under conditions such that said derviatized recognition binding moieties react to from a covalent linkage with the PEG linker, thereby producing a population of bispecific molecules.

30. The method of claim 29, wherein said molecule is desired to be reduced in amount in the circulation of a mammal.

31. The method of claim 29, wherein said one or more recognition binding moieties are derivatized by a method comprising thiolating said one or more recognition binding moieties with a thiol specific derivatizing agent.

32. The method of claim 31, wherein said thiol specific derivatizing agent is selected from a group consisting of succinimidyl-3-(2-pyridylthio-propionate) (SPDP), or succinimidyl acetylthioacetate(SATA).

33. The method of claim 29, wherein said one or more recognition binding moieties are derivatized by a method comprising modifying said one or more recognition binding moieties with a hydrazine or aldehyde modification reagent.

34. The method of claim 33, wherein said hydrazine modification reagent is succinimidyl 6-hydrazinonicotinate acetone hydrazone (SANH) or succinimidyl 4-formyl benzoate (SFB).

35. The method of claim 29, wherein said bifunctional poly-(ethylene) glycol (PEG) molecule is a heterobifunctional poly-(ethylene)glycol (PEG), having the formula X-PEG-Y.

36. The method of claim 35, wherein said heterobifunctional poly-(ethylene)glycol (PEG) is selected from the group consisting of NHS-PEG maleimide, NHS-PEG-vinylsulfone, bis-hydrazide-PEG, aldehyde-PEG-NHS, and bis-hydrazine-PEG.

37. A method of producing a population of bispecific molecules said method comprising:
(a) contacting an anti-CR1 antibody with NHS-poly-(ethylene)glycol (PEG)-maleimide, such that the anti-CR1 antibody is derivatized at one or more sites with the NHS functional group of the NHS-PEG-maleimide;
(b) contacting a recognition binding moiety with N-succinimidyl-S-acetyl-thioacetate (SATA), such that the antigen recognition binding moiety is derivatized to contain one or more free thiol, and wherein said recognition binding moiety binds a molecule;
(c) combining the poly-(ethylene)glycol (PEG)-derivatized anti-CR1 antibody produced in step (a) with the thiol derivatized recognition binding moiety produced in step (b);
thereby producing a population of bispecific molecules.

38. The method of claim 37, wherein said recognition binding moiety binds the protective antigen (PA) protein of Bacillus anthracis (Anthrax).

39. The method of claim 37, wherein said molecule is an autoimmune antigen or is an antigen of an infectious disease agent.

40. The method of claim 37, wherein said molecule is the protective antigen (PA) protein of Bacillus anthracis (Anthrax).

41. The method of claim 37, wherein said step (c) is carried out by a method comprising mixing said PEG-derivatized anti-CR1 antibody and said SATA-derivatized recognition binding moiety at a molar ratio of 1:1.

42. The method of claim 37, wherein said step (c) is carried out by a method comprising mixing said PEG-derivatized anti-CR1 antibody and said SATA-derivatized recognition binding moiety at a molar ratio of 2:1.

43. The method of claim 37 or 29, further comprising isolating and purifying said population of bispecific molecules.

44. The method of claim 43, wherein said method for isolating and purifying said population of bispecific molecules comprises size exclusion chromatography.

45. The method of claim 37, wherein said anti-CR1 antibody is derivatized with NHS-PEG-maleimide at a molar ratio of 1:4, anti-CR1 antibody:NHS-PEG-maleimide.

46. The method of claim 37, wherein said anti-CR1 antibody is derivatized with NHS-PEG-maleimide at a molar ratio of 1:8, anti-CR1 antibody NHS-PEG-maleimide.

47. The method of claim 37, wherein said anti-CR1 antibody is derivatized with NHS-PEG-maleimide at a molar ratio of 1:16, anti-CR1 antibody:NHS-PEG-maleimide.

48. The method of claim 37, wherein said recognition binding moiety is derivatized with N-succinimidyl-S-acetyl-thioacetate (SATA) at a molar ratio of 1:4, recognition binding moiety:SATA.

49. The method of claim 37, wherein said recognition binding moiety is derivatized with N-succinimidyl-S-acetyl-thioacetate (SATA) at a molar ratio of 1:8, recognition binding moiety:SATA.

50. The method of claim 37, wherein said recognition binding moiety is derivatized with N-succinimidyl-S-acetyl-thioacetate (SATA) at a molar ratio of 1:16, recognition binding moiety:SATA.

51. A population of bispecific molecules produced by the method of claim 29 or 37.

52. A method of producing a population of antibodies that bind a C3b-like receptor comprising a polyethylene glycol linker, said method comprising contacting the antibodies with a polyethylene glycol linker, such that the antibodies are derivatized at one or more sites with the polyethylene glycol linker, thereby producing a population of PEG-derivatized antibodies.

53. The method of claim 52, wherein said PEG-derivatized antibodies bind the C3b-like receptor with an activity at least 50% of the antibodies that contained no PEG derivatives.

54. The population of the PEG-derivatized antibodies produced by the method of claim 52.

55. A pharmaceutical composition comprising a therapeutically effective amount of the bispecific molecule of claim 1, said amount being effective for treating a mammal having an undesirable condition associated with the presence of said molecule in the circulation of a mammal, and a pharmaceutically acceptable carrier.

56. A kit comprising:
(a) a first container comprising a polyethylene glycol-derivatized anti-CR1 antibody;
(b) a second container comprising a recognition binding moiety, said recognition binding moiety being other than an anti-CR1 antibody; and
(c) a third container comprising a derivatizing agent suitable to derivatize said one or more recognition binding moieties.

57. The bispecific molecule of claim 21, wherein said one or more second recognition binding moieties are antibodies, and wherein said bispecific molecule is oxidized at one or more carbohydrate moieties within the Fc region of the first or second recognition binding moieties, and wherein said oxidized carbohydrate is the site at which a PEG linker is derivatized.

58. The bispecific molecule of claim 10, wherein the first or second recognition binding moieties is an antibody and wherein the PEG linker derivatizes one or more oxidized carbohydrate moieties within the Fc region of the first or second recognition binding moieties.

59. The bispecific molecule of claim 57 or 58, wherein said oxidized carbohydrate moieties are oxidized chemically or enzymatically.

60. The bispecific molecule of claim 1, wherein said first recognition binding moiety binds CR1.

61. A method of treating a disorder in a mammal comprising administering a therapeutically effective amount of the bispecific molecule of claim 1, wherein said disorder is associated with the presence of said molecule in the circulation of the mammal.

62. A method of producing a population of bispecific molecules said method comprising:
(a) contacting an anti-CR1 antibody with NHS-poly-(ethylene)glycol (PEG)-benzaldehyde, such that the anti-CR1 antibody is derivatized at one or more sites with the NHS functional group;
(b) contacting a recognition binding moiety with C6 4-hydrazino-nicotinamide acetone hydrazone such that the antigen recognition binding moiety is derivatized, and wherein said recognition binding moiety binds a molecule; and
(c) combining the poly-(ethylene)glycol (PEG)-derivatized anti-CR1 antibody produced in step (a) with the hydrazone derivatized recognition binding moiety produced in step (b);
thereby producing a population of bispecific molecules.

63. The bispecific molecule of claim 1, wherein the PEG linker is NHS-poly-(ethylene)glycol (PEG)-benzaldehyde.

64. A population of bispecific molecules produced by the method of claim 62.

65. A bispecific molecule comprising:
(a) a first recognition binding moiety that binds a C3b-like receptor; and
(b) one or more second recognition binding moieties that binds a molecule; said molecule being other than a C3b-like receptor; wherein said first recognition binding moiety is cross-linked via an NHS-poly-(ethylene) glycol (PEG)-benzaldehyde linker to the second recognition binding moieties.

66. The bispecific molecule of claim 65, wherein the first recognition binding moiety is a deimmunized anti-CR1 monoclonal antibody.

67. The bispecific molecule of claim 66, wherein the deimmunized anti-CR1 monoclonal antibody is H9.

68. The method of claim 62, wherein said recognition binding moiety binds the protective antigen (PA) protein of Bacillus anthracis (Anthrax).

69. The method of claim 62, wherein said molecule is an autoimmune antigen or is an antigen of an infectious disease agent.

70. The method of claim 62, wherein said molecule is the protective antigen (PA) protein of Bacillus anthracis (Anthrax).

71. A pharmaceutical composition comprising a therapeutically effective amount of the bispecific molecule of any one of claims 65-67, said amount being effective for treating a mammal having an undesirable condition associated with the presence of said molecule in the circulation of a mammal, and a pharmaceutically acceptable carrier.

72. A method of treating a disorder in a mammal comprising administering a therapeutically effective amount of the bispecific molecule of any one of claims 65-67, wherein said disorder is associated with the presence of said molecule in the circulation of the mammal.

73. A compound of the formula: 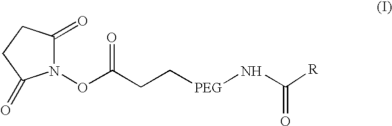
or a pharmaceutically acceptable salt thereof, wherein R is phenyl, naphthyl, or aromatic heterocycle, any of which is substituted with at least one —C(O)H or —NH—NH₂ group.

74. The compound of claim 73 having the formula: 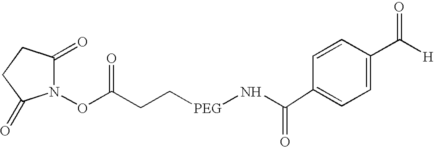
or a pharmaceutically acceptable salt thereof.

75. The compound of claim 73 having the formula:

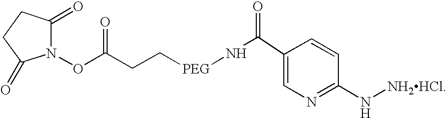

76. An antibody derivatized with the compound of any one of claims 73-75.

77. The method of claim 29, wherein said linker is a compound of the formula: [chemical structure]
